SEQUENCE LISTING (16 sequences)

SEQ ID NO: 1 - protein, 606 aa, Artificial Sequence
  HB0028 heavy chain
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSGGGGSGQVQLVQSGAEVKKPGASVKVSCKASGYAFTGYTIHWVRQAPGQRLEWMGWFYPGSGTLKYSEKFQGRVTITRDKSLSTAYMELSSLRSEDTAVYYCARHGTGTLMAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 136 aa, Artificial Sequence
  TGF-beta RII ECD24-159
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 3 - protein, 21 aa, Artificial Sequence
  GS linker
GGGGSGGGGSGGGGSGGGGSG

SEQ ID NO: 4 - protein, 120 aa, Artificial Sequence
  antibody heavy chain variable region of HB0028 and HB0029
QVQLVQSGAEVKKPGASVKVSCKASGYAFTGYTIHWVRQAPGQRLEWMGWFYPGSGTLKYSEKFQGRVTITRDKSLSTAYMELSSLRSEDTAVYYCARHGTGTLMAMDYWGQGTLVTVSS

SEQ ID NO: 5 - protein, 329 aa, Artificial Sequence
  antibody heavy chain constant region of HB0028 and HB0029
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 606 aa, Artificial Sequence
  heavy chain of HB0029
QVQLVQSGAEVKKPGASVKVSCKASGYAFTGYTIHWVRQAPGQRLEWMGWFYPGSGTLKYSEKFQGRVTITRDKSLSTAYMELSSLRSEDTAVYYCARHGTGTLMAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 7 - protein, 219 aa, Artificial Sequence
  light chain 400085
DVVMTQTPLSLSVTPGQPASISCKSSQSLANSYGNTYLSWYLHKPGQSPQLLIYGISNRFSGVPDRFSGSGSGTDFTLKISRVEAEDVGVYYCLQGTHQPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 112 aa, Artificial Sequence
  variable region of light chain 400085
DVVMTQTPLSLSVTPGQPASISCKSSQSLANSYGNTYLSWYLHKPGQSPQLLIYGISNRFSGVPDRFSGSGSGTDFTLKISRVEAEDVGVYYCLQGTHQPPTFGQGTKLEIK

SEQ ID NO: 9 - protein, 107 aa, Artificial Sequence [sequence omitted]

SEQ ID NO: 10 - DNA, 37 bp, Artificial Sequence
  forward primer
aggagatgaccaagaaccaggtaagtttgacctgcct

SEQ ID NO: 11 - DNA, 41 bp, Artificial Sequence
  reverse primer
accgcgagagcccggggagcgggggcttgccggccgtcgca

SEQ ID NO: 12 - protein, 8 aa, Artificial Sequence
  CDR1 of PD-L1 antibody heavy chain
GYAFTGYT

SEQ ID NO: 13 - protein, 8 aa, Artificial Sequence
  CDR2 of PD-L1 antibody heavy chain
FYPGSGTL

SEQ ID NO: 14 - protein, 13 aa, Artificial Sequence
  CDR3 of PD-L1 antibody heavy chain
ARHGTGTLMAMDY

SEQ ID NO: 15 - protein, 11 aa, Artificial Sequence
  CDR1 of PD-L1 antibody light chain
QSLANSYGNTY

SEQ ID NO: 16 - protein, 9 aa, Artificial Sequence
  CDR3 of PD-L1 antibody light chain
LQGTHQPPT

(19) United States
(12) Patent Application Publication
(10) Pub. No.: US 2024/0026004 A1
(43) Pub. Date: Jan. 25, 2024

(54) ANTI-PD-L1/TGF-ß BIFUNCTIONAL ANTIBODY AND USE THEREOF

(71) Applicants: SHANGHAI HUAOTA BIOPHARMACEUTICAL CO., LTD., Pudong New Area, Shanghai (CN); HUABO BIOPHARM (SHANGHAI) CO., LTD., Pudong New Area, Shanghai (CN)

(72) Inventors: Xiaoyue WEI, Shanghai (CN); Xiangyang ZHU, Shanghai (CN); Xianfei PAN, Shanghai (CN); Xue LI, Shanghai (CN); Xiaochen REN, Shanghai (CN)

(21) Appl. No.: 18/039,733

(22) PCT Filed: Dec. 1, 2021
(86) PCT No.: PCT/CN2021/134824

§ 371 Date: Jun. 1, 2023

(30) Foreign Application Priority Data
Dec. 2, 2020 (CN) 202011401649.6

(51) Int. Cl. C07K 16/28; C07K 16/22; C07K 14/71; A61P 35/00

(52) U.S. Cl. CPC C07K 16/2827; C07K 16/22; C07K 14/71; A61P 35/00; C07K 2317/31; C07K 2317/92; C07K 2317/76; A61K 2039/505

(57) ABSTRACT

Provided in the present application are an anti-PD-L1/TGF-β bispecific antibody and the use thereof. Specifically, provided in the present application is a bifunctional antibody, which comprises: (a) an anti-PD-L1 antibody or element; and (b) an anti-TGF-β antibody or element connected to the anti-PD-L1 antibody or element. The bifunctional antibody of the present application can simultaneously bind to TGF-β and PD-L1, thereby exerting a therapeutic effect on TGF-β and PD-L1-positive tumor cells.

### FIELD OF THE INVENTION

The present application relates to the field of tumor immunology, and more specifically, to an anti-PD-L1/TGF-β bifunctional antibody and a use thereof.

### BACKGROUND OF THE INVENTION

Cancer is the second leading cause of human death after cardiovascular disease. According to the World Health Organization's 2018 Global Cancer Report, there were 18.1 million new cancer cases and 9.6 million deaths worldwide in 2018, which is equivalent to 1 in 6 deaths from cancer. Among them, lung cancer, breast cancer, colorectal cancer, and gastric cancer are among the top cancers in terms of incidence and death (FIG. 1). The data also show that about half of new cases and deaths worldwide occur in Asia, with China, as a populous country, accounting for a large portion. For a long time, most cancer treatments can only temporarily prolong the survival of patients. A diagnosis of cancer is like a death sentence, which makes people reluctant to talk about it. Pharmaceutical companies around the world continue to invest in the development of anti-cancer drugs, and new drugs on the market are continuously upgraded, ranging from chemotherapy and radiotherapy which are considered as pyrrhic victories, to molecularly targeted drugs and combination of chemotherapy and targeted drugs against tumor-related antigens, as well as the immunotherapy that is very hot nowadays. The pathogenesis of cancer is becoming clearer, and immunosuppression in the tumor microenvironment is an important factor in the formation of tumors. Immunotherapy is the use of the immune system to kill tumor cells by normalizing the immunity within the tumor through modulators or by artificially importing immune tools. Amazing advances in tumor immunotherapy are changing the standard of care for many cancer types, and curing a cancer or transforming it into a manageable chronic disease has become the goal of cancer treatment in such a new era.

At present, a large number of new products under study and companies have entered the field of tumor immunotherapy, including immunomodulators, CAR-T cells and bispecific antibodies. Both activating and inhibitory molecules are expressed on immune cells to ensure the immune homeostasis of the body. Tumor immune escape refers to the phenomenon that tumor cells escape from the identification and attack of the body immune system by means of multiple mechanisms, so as to survive and proliferate in the body. Immune checkpoints such as CTLA-4 and PD-1 are one way of tumor immune escape. PD-L1 is mainly overexpressed on the surface of various tumor cells and binds to PD-1 molecules on T cells to induce T cell apoptosis, thus helping tumor immune escape. In recent years, 10 types of monoclonal antibody drugs targeting PD-1 or PD-L1 have been launched one after another with obvious clinical effects, among which Keytruda (Pembrolizumab) and Opdivo (Nivolumab) have successfully entered the Top 10 list of global drug sales.

TGF-β is mainly expressed and secreted by the immune system (including TGF-β/2/3), and can regulate the growth, proliferation, differentiation, migration and apoptosis of cells after binding to receptors TGF-β R (including RI/RII/RIII), which affects embryonic organ development and organism immunity and thus has important physiological functions. Three subtypes of TGF-β1, TGF-β2, and TGF-β3 can bind to receptors on the cell surface. TGF-βRI does not directly bind TGF-β, RIII can bind TGF-β, but its sugar modification is too complex. TGF-βRII has extremely high affinity for TGF-β1/3 (about 5 pM) and lower affinity for TGF-β2 (about 6 nM). TGF-β plays a very important and dual role in the occurrence and development of tumors. In the early stage of tumors, TGF-β can regulate the expression of several apoptotic genes to induce apoptosis of tumor cells; while in the late stage of tumors, most tumor cells secrete a large amount of TGF-β. Once the level of TGF-β is too high, it will be transformed into a tumor-promoting factor, which can inhibit T and NK cells, promote regulatory T cells, promote tumor angiogenesis, and promote the transformation of epithelial cells to mesenchymal cells and the like, thus promoting the metastasis and development of the tumor. It has been reported that the abnormal regulation of TGF-β signaling pathway-related genes is one of the reasons for PD-1 antibody resistance. Therefore, TGF-β-targeted drugs have also become an important direction for the development of anti-cancer drugs.

The PD-1/PD-L1 inhibitors have been preliminarily employed in the treatment of tumors, but their clinical average efficiency is from 20% to 30%, and there is still much room for improvement in the indications of PD-L1 inhibitors. More and more data show that PD-1/PD-L1 combined with chemotherapy, targeted therapy, or other immunotherapy (e.g., CTLA4 inhibitors) is effective in improving the objective remission rates and can benefit more patients. The tissue structure of tumors is very complex, and the expression level of PD-L1 in tumors is one of the reasons for the ineffectiveness of PD-1/PD-L1 inhibitors. Moreover, there are a variety of immunosuppressive cells (such as MDSC, regulatory T cells, tumor-associated macrophages) and inflammatory related factors (such as IL-6, IL-10, TGF-β) in the microenvironment of tumors, which jointly promote tumor immune escape, tumor growth and metastasis. Therefore, in addition to the immune checkpoint modulators that “remove shackles on T cells”, the “T cell openers” that target to reshape the tumor microenvironment of inflammatory related factors are also the important direction for the development of anti-cancer drugs.

TGF-β is an important target for tumor microenvironment regulation, however, the TGF-β receptor has an extremely high affinity for TGF-β, which poses a great challenge to the development of antibodies. The antibody affinity must be high enough to compete with the receptor for binding TGF-β, while if the affinity is too high, off-target binding is likely to occur in vivo. Drugs must be developed to ensure the safety for treatment. For this, affinity and dose can only be down-regulated, and the effectiveness of drugs is forced to be compromised. Hence, even though major pharmaceutical companies have entered the field of TGF-β-targeted drugs, no TGF-β-related drugs have been marketed so far. Therefore, it is of great significance to develop dual-target therapeutics that block both PD-L1 and TGF-β molecules. The PD-L1 binding arm of the dual antibody can be targeted to tumor tissues, improving the targeting efficiency of the antibody and reducing the off-target toxic side effects. Although bifunctional antibodies are the direction for the development of antibody drugs, many challenges still exist, such as preclinical evaluation models, low expression levels, poor stability, complex processes, and high variability in quality control. Therefore, it is urgent in this field to develop an anti-tumor dual antibody with good specificity, good therapeutic efficacy and easy preparation.

### SUMMARY OF THE INVENTION

The present application aims to provide an anti-PD-L1/TGF-β bifunctional antibody and a use thereof.

In a first aspect of the present application, provided is a bifunctional antibody, which includes:
(a) an anti-PD-L1 antibody or element; and
(b) an anti-TGF-β antibody or element connected to the anti-PD-L1 antibody or element.

In some embodiments, the anti-PD-L1 antibody or element is connected to the anti-TGF-β antibody or element through a connecting peptide.

In some embodiments, the anti-TGF-β antibody or element is connected to a region of the anti-PD-L1 antibody selected from the group consisting of a heavy chain variable region, a heavy chain constant region, a light chain variable region, or a combination thereof.

In some embodiments, the anti-TGF-β antibody or element is connected to an initial terminal of the heavy chain variable region of the anti-PD-L1 antibody.

In some embodiments, the anti-TGF-β antibody or element is connected to a terminal end of the heavy chain constant region of the anti-PD-L1 antibody.

In some embodiments, the antibody is selected from the group consisting of a nanobody, a single-stranded antibody, and a double-stranded antibody.

In some embodiments, the antibody is selected from the group consisting of an animal-derived antibody (such as murine-derived antibodies), a chimeric antibody, and a humanized antibody.

In some embodiments, the humanized antibody includes a full human antibody.

In some embodiments, the element includes an extracellular region of a ligand, a receptor, or a protein.

In some embodiments, the anti-TGF-β element includes an extracellular region of a TGF-β receptor.

In some embodiments, the TGF-β receptor includes TGF-βRI, TGF-βRII, and TGF-βRIII, e.g., it may be TGF-βRII.

In some embodiments, in the bifunctional antibody, the number of the anti-TGF-β element is 1 to 4, e.g., it may be 2.

In some embodiments, the bifunctional antibody is a homodimer.

In some embodiments, the bifunctional antibody has a structure represented by formula Ia or Ib from N-terminus to C-terminus:

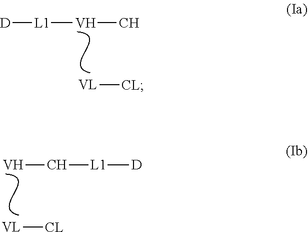

wherein,
“-” represents a peptide bond;
“ [symbol] ” represents a disulfide bond;
D is an anti-TGF-β element;
L1 is none or an adaptor element;
VH represents the heavy chain variable region of the anti-PD-L1 antibody;
CH represents the heavy chain constant region of the anti-PD-L1 antibody;
VL represents the light chain variable region of the anti-PD-L1 antibody;
CL represents the light chain constant region of the anti-PD-L1 antibody;
wherein, the bifunctional antibody has an activity of simultaneously binding to PD-L1 and TGF-β.

In some embodiments, the anti-TGF-β element includes a TGF-βRII extracellular region, e.g., the amino acid sequence of the TGF-βRII extracellular region is as set forth in SEQ ID NO: 2.

In some embodiments, the adaptor element is a GS connecting peptide, e.g., the amino acid sequence of the GS connecting peptide is as set forth in SEQ ID NO: 3.

In some embodiments, the heavy chain variable region (VH) of the anti-PD-L1 antibody includes the following three complementary determining regions (CDRs):
a CDR1 as set forth in SEQ ID NO: 12,
a CDR2 as set forth in SEQ ID NO: 13, and
a CDR3 as set forth in SEQ ID NO: 14; and/or
the light chain variable region (VL) of the anti-PD-L1 antibody includes the following three complementary determining regions (CDRs):
a CDR1′ as set forth in SEQ ID NO: 15,
a CDR2′ with an amino acid sequence of GIS, and
a CDR3′ as set forth in SEQ ID NO: 16.

In some embodiments, the amino acid sequence of the heavy chain variable region (VH) of the anti-PD-L1 antibody is as set forth in SEQ ID NO: 4.

In some embodiments, the amino acid sequence of the heavy chain constant region of the anti-PD-L1 antibody is as set forth in SEQ ID NO: 5.

In some embodiments, the amino acid sequence of the light chain variable region (VL) of the anti-PD-L1 antibody is as set forth in SEQ ID NO: 8.

In some embodiments, the amino acid sequence of the light chain constant region of the anti-PD-L1 antibody is as set forth in SEQ ID NO: 9.

In some embodiments, the bifunctional antibody has a structure represented by formula Ia.

In some embodiments, the bifunctional antibody is a homodimer with a structure represented by formula Ia.

In some embodiments, the bifunctional antibody is a double-stranded antibody.

In some embodiments, the bifunctional antibody has a heavy chain (H chain) and a light chain (L chain).

In some embodiments, the H chain of the bifunctional antibody has an amino acid sequence as set forth in SEQ ID NO: 1.

In some embodiments, the L chain of the bifunctional antibody has an amino acid sequence as set forth in SEQ ID NO: 7.

In some embodiments, the antibody is in a form of a drug conjugate.

In some embodiments, the bifunctional antibody is conjugated with a tumor-targeted marker conjugate.

In some embodiments, the bifunctional antibody further includes (e.g., is conjugated with) a detectable marker, targeting label, drug, toxin, cytokine, radionuclide, enzyme, or a combination thereof.

In some embodiments, the bifunctional antibody further includes an active fragment and/or a derivative of the bifunctional antibody, wherein, the active fragment and/or the derivative retains 70-100% (such as 70%, 75%, 80%, 85%, 90%, 95%, 96%, 97%, 98%, 99%, 100%) of the anti-PD-L1 activity and 70-100% of the anti-TGF-β activity of the bifunctional antibody.

In some embodiments, the derivative of the antibody is a sequence obtained after deletion, insertion and/or substitution of one or several amino acids of the bifunctional antibody of the present application and maintaining at least 85% identity.

In some embodiments, the derivative of the antibody has at least 86%, 87%, 88%, 89%, 90%, 91%, 92%, 93%, 94%, 95%, 96%, 97%, 98%, 99% sequence identity to the bifunctional antibody of the present application.

In some embodiments, the substitution is conservative substitution.

In a second aspect of the present application, provided is an isolated polynucleotide (a composition), which encodes the bifunctional antibody in the first aspect of the present application.

In some embodiments, the polynucleotide (composition) includes a polynucleotide encoding the L chain of the bifunctional antibody.

In some embodiments, the polynucleotide (composition) includes a polynucleotide encoding the H chain of the bifunctional antibody.

In some embodiments, in the polynucleotide (composition), the ratio of the polynucleotide encoding the L chain to the polynucleotide encoding the H chain is 1:1.

In some embodiments, in the polynucleotide (composition), the polynucleotide encoding the L chain and the polynucleotide encoding the H chain exist independently.

In a third aspect of the present application, provided is a vector containing the polynucleotide in the second aspect of the present application.

In some embodiments, the vector includes all the polynucleotides of the polynucleotides in the second aspect of the present application.

In some embodiments, the vector includes any one of the polynucleotides in the second aspect of the present application, respectively.

In some embodiments, the vector is an expression vector.

In some embodiments, the vector includes plasmids, phages, yeast plasmids, plant cell viruses, mammalian cell viruses such as adenoviruses, retroviruses, or other vectors.

In another aspect of the present application, provided is a vector composition which includes a vector containing any one polynucleotide of the polynucleotide composition in the second aspect of the present application.

In some embodiments, the vector composition includes a vector containing the polynucleotide encoding the L chain and a vector containing the polynucleotide encoding the H chain.

In a fourth aspect of the present application, provided is a genetically engineered host cell which contains the vector in the third aspect of the present application or is integrated with the polynucleotide in the second aspect of the present application within its genome.

In some embodiments, the host cell includes prokaryotic cells or eukaryotic cells.

In some embodiments, the host cell is selected from the group consisting of E. coli, yeast cells, and mammalian cells.

In some embodiments, the host cell includes CHO cells.

In a fifth aspect of the present application, provided is a method for preparing the bifunctional antibody in the first aspect of the present application, which includes the following steps:
(i) culturing the host cell in the fourth aspect of the present application under suitable conditions to obtain a mixture containing the bifunctional antibody in the first aspect of the present application; and
(ii) purifying and/or isolating the mixture obtained in step (i) to obtain the bifunctional antibody in the first aspect of the present application.

In some embodiments, a target antibody can be obtained through purification by isolating over a Protein A affinity column.

In some embodiments, the purity of the target antibody isolated through purification is greater than 95%, greater than 96%, greater than 97%, greater than 98%, greater than 99%, and the purity can be 100%.

In a sixth aspect of the present application, provided is an immunoconjugate, which contains:
(a) the bifunctional antibody in the first aspect of the present application; and
(b) a conjugating part selected from the group consisting of a detectable marker, drug, toxin, cytokine, radionuclide, or enzyme, gold nanoparticle/nanorod, nanomagnetic particle, virus coat protein or VLP, or a combination thereof.

In some embodiments, the antibody is partially conjugated with the conjugating part through a chemical bond or an adaptor.

In some embodiments, the radionuclide includes:
(i) an isotope for diagnosis selected from the group consisting of Tc-99m, Ga-68, F-18, I-123, I-125, I-131, In-111, Ga-67, Cu-64, Zr-89, C-11, Lu-177, Re-188, or a combination thereof; and/or
(ii) a therapeutic isotope selected from the group consisting of Lu-177, Y-90, Ac-225, As-211, Bi-212, Bi-213, Cs-137, Cr-51, Co-60, Dy-165, Er-169, Fm-255, Au-198, Ho-166, 1-125, I-131, Ir-192, Fe-59, Pb-212, Mo-99, Pd-103, P-32, K-42, Re-186, Re-188, Sm-153, Ra223, Ru-106, Na24, Sr89, Tb-149, Th-227, Xe-133 Yb-169, Yb-177, or a combination thereof.

In some embodiments, the conjugating part is a drug or a toxin.

In some embodiments, the drug is a cytotoxic drug.

In some embodiments, the cytotoxic drug is selected from the group consisting of antitubulin drugs, DNA minor groove binding reagents, DNA replication inhibitors, alkylating agents, antibiotics, folic acid antagonists, antimetabolites, chemosensitizers, topoisomerase inhibitors, vinca alkaloids, or a combination thereof.

Examples of particularly useful cytotoxic drugs include, for example, DNA minor groove binding reagents, DNA alkylating agents, and tubulin inhibitors, typical cytotoxic drugs including, e.g., auristatins, camptothecins, duocarmycins, etoposides, maytansines and maytansinoids (e.g., DM1 and DM4), taxanes, benzodiazepines or benzodiazepine containing drugs (e.g., pyrrolo[1,4]benzodiazepines (PBDs) indolinobenzodiazepines, and oxazolidinobenzodiazepines), vinca alkaloids, or a combination thereof.

In some embodiments, the toxin is selected from the group consisting of: Auristatins (e.g., auristatin E, auristatin F, MMAE, and MMAF), aureomycin, maytansinoid, ricin, ricin A-chain, combretastatin, docamicin, dolastatin, adriamycin, daunorubicin, paclitaxel, cisplatin, ccl065, ethidium bromide, mitomycin, etoposide, tenoposide, vincristine, vinblastine, colchicine, dihydroxy anthracin dione, actinomycin, diphtheria toxin, pseudomonas exotoxin (PE) A, PE40, abrin, abrin A chain, modeccin A chain, α-sarcina, gelonin, mitogellin, retstrictocin, phenomycin, enomycin, curicin, crotin, calicheamicin, Sapaonaria officinalis inhibitors, glucocorticoids, or a combination thereof.

In some embodiments, the conjugating part is a detectable marker.

In some embodiments, the conjugate is selected from the group consisting of fluorescent or luminescent markers, radioactive markers, MRI (Magnetic resonance imaging) or CT (computed tomography) contrast media, or enzymes capable of producing detectable products, radionuclides, biotoxins, cytokines (e.g., IL-2, etc.), antibodies, antibody Fc fragments, antibody scFv fragments, gold nanoparticles/nanorods, virions, lipidosomes, nanomagnetic particles, prodrug activating enzymes (e.g., DT-diaphorase (DTD) or biphenyl hydrolase-like protein (BPHL)), chemotherapeutic agents (e.g., cisplatin) or any form of nanoparticles.

In some embodiments, the immunoconjugate contains a multivalent (e.g., divalent) bifunctional antibody in the first aspect of the present application.

In a seventh aspect of the present application, provided is a pharmaceutical composition containing:
(I) the bifunctional antibody in the first aspect of the present application, or the immunoconjugate in the sixth aspect of the present application; and
(II) a pharmaceutically acceptable carrier.

In some embodiments, the pharmaceutical composition also contains an additional anti-tumor agent, such as a cytotoxic drug.

In some embodiments, the pharmaceutical composition is in a unit dosage form.

In some embodiments, the anti-tumor agent includes paclitaxel, Doxorubicin, cyclophosphamide, Axitinib, Lenvatinib, or Pembrolizumab.

In some embodiments, the anti-tumor agent can be present in separate packages with the bifunctional antibody, or the anti-tumor agent can be conjugated with the bifunctional antibody.

In some embodiments, the dosage form of the pharmaceutical composition includes gastrointestinal administration dosage forms or parenteral administration dosage forms.

In some embodiments, the parenteral administration dosage forms include intravenous injection, intravenous infusion, subcutaneous injection, local injection, intramuscular injection, intratumoral injection, intra-abdominal injection, intracranial injection, or intracavitary injection.

In an eighth aspect of the present application, provided is a use of the bifunctional antibody in the first aspect of the present application or the immunoconjugate in the sixth aspect of the present application in the preparation of (a) a detection reagent or a kit; and/or (b) a pharmaceutical composition for preventing and/or treating cancers or tumors.

In some embodiments, the tumor is selected from the group consisting of hematologic tumors, solid tumors, or a combination thereof.

In some embodiments, the tumor is selected from the group consisting of ovarian cancer, colon cancer, rectal cancer, melanoma (e.g., metastatic malignant melanoma), kidney cancer, bladder cancer, breast cancer, liver cancer, lymphoma, hematologic malignancies, head and neck cancer, glioma, gastric cancer, nasopharyngeal cancer, laryngeal cancer, cervical cancer, uterine myoma, and osteosarcoma. Examples of other cancers that can be treated by the method of the present application include: bone cancer, membrane adenocarcinoma, skin cancer, prostate cancer, skin or intraocular malignant melanoma, uterine cancer, anal cancer, testicular cancer, fallopian tube cancer, endometrial cancer, vaginal cancer, vulvar cancer, Hodgkin's disease, Non-Hodgkin's lymphoma, esophageal cancer, small intestine cancer, endocrine system cancer, thyroid cancer, parathyroid cancer, adrenocortical carcinoma, soft tissue sarcoma, urethral carcinoma, penile cancer, chronic or acute leukemia including acute myeloid leukemia, chronic myeloid leukemia, acute lymphoblastic leukemia, chronic lymphocytic leukemia, pediatric solid tumors, lymphocytic lymphoma, bladder cancer, renal or ureteral cancer, renal pelvic carcinoma, central nervous system (CNS) tumors, primary CNS lymphoma, tumor angiogenesis, spinal tumors, brainstem gliomas, pituitary adenoma, Kaposi's sarcoma, epidermoid carcinoma, squamous cell carcinoma, T cell lymphoma, environmentally induced cancers including asbestos-induced cancers, and combinations of these cancers.

In some embodiments, the tumor is rectal cancer, non-small cell lung cancer, melanoma, bladder cancer, or a combination thereof.

In some embodiments, the tumor is tumors with high expression of PD-L1 and/or TGF-β.

In some embodiments, the drugs or preparations are used to prepare drugs or preparations for preventing and/or treating diseases related to PD-L1 and/or TGF-β (positive for expression).

In some embodiments, the antibody is in a form of an antibody-drug conjugate (ADC).

In some embodiments, the detection reagent or kit is used for the diagnosis of PD-L1 and/or TGF-β related diseases.

In some embodiments, the detection reagent or kit is used for the detection of PD-L1 and/or TGF-β protein in the samples.

In some embodiments, the detection reagent is a detection plate.

In a ninth aspect of the present application, provided is a method for treating tumors, which includes a step of administering to a subject in need the bifunctional antibody in the first aspect of the present application, or the immunoconjugate in the sixth aspect of the present application, or the pharmaceutical composition in the seventh aspect of the present application, or a combination thereof.

Other aspects and advantages of the present application can be readily perceived by those skilled in the art from the following detailed description. In the following detailed description, only exemplary embodiments of the present application are shown and described. As will be recognized by those skilled in the art, the content of the present application enables those skilled in the art to make changes to the disclosed specific embodiments without departing from the spirit and scope of the invention involved in the present application. Correspondingly, the drawings and descriptions in the specification of the present application are merely exemplary, rather than restrictive.

### BRIEF DESCRIPTION OF THE DRAWINGS

The specific features of the invention involved in the present application are as set forth in the appended claims. The characteristics and advantages of the invention involved in the present application can be better understood by referring to the exemplary embodiments described in detail below and the accompanying drawings. A brief description of the drawings is as below:

FIG. 1 shows the types of cancers with the highest number of incidences and deaths worldwide in 2018.

FIG. 2 shows the structural schematic diagrams of HB0028 and HB0029.

FIG. 3 shows the purification results of Protein A affinity column detected by SDS-PAGE. Wherein, M represents Protein Molecular Weight Marker.

FIG. 4 shows the binding activities of HB0028 and HB0029 for human TGF-β1.

FIG. 5 shows the binding activities of HB0028 and HB0029 for human TGF-β3.

FIG. 6 shows the binding activities of HB0028 and HB0029 for human PD-L1.

FIG. 7 shows the binding activities of HB0028 and HB0029 for dual targets of PD-L1 and TGF-β.

FIG. 8 shows the effects of HB0028 and HB0029 for restoring T cell activation.

FIG. 9 shows the inhibitory effects of HB0028 and HB0029 on the TGF-β/SMAD signaling pathway.

FIG. 10 shows the anti-tumor effect of the antibody in the human melanoma A375 combined PBMC subcutaneous xenotransplanted tumor model.

FIG. 11 shows the anti-tumor effect of the antibody in the human breast cancer MDA-MB-231 combined PBMC subcutaneous xenotransplanted tumor model.

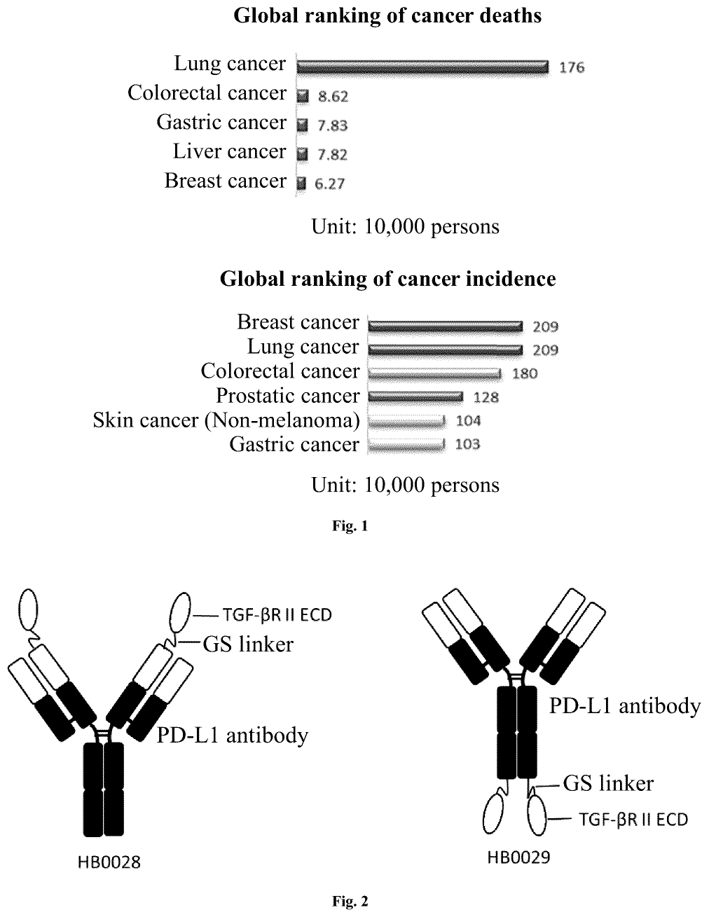

### DETAILED DESCRIPTION OF THE EMBODIMENTS

After intensive and extensive studies, the applicant first constructed an anti-PD-L1/TGF-β bifunctional antibody. Specifically, on the basis of the PD-L1 humanized monoclonal antibody HB0023 (see Chinese patent application CN201910258153.9) independently developed by the applicant, the extracellular region (ECD) of human TGF-βR II was connected to the N-terminus or C-terminus of the heavy chain of the monoclonal antibody through a flexible GS linker, to obtain a dual-target fusion monoclonal antibody that binds to PD-L1 and TGF-β molecules both with 2 valence, which were respectively named as HB0028 and HB0029, with their structural schematic diagrams shown in FIG. 2.

In the previous study, the applicant has identified a variety of bispecific antibodies with different structures and different linkage modes, and finally obtained bispecific antibodies HB0028 and HB0029 with the best technical effect by comparing their target binding activity, blocking activity, signaling pathway inhibition function, product purity and/or stability, and determined the amino acid sequence and gene sequence. Wherein, the structural stability of HB0028 was better than that of HB0029, and it could retain the binding activity of the TGF-βRII extracellular region better. Subsequently, the plasmid carrying the HB0028 gene was transfected into CHO host cells, and cell line that could express HB0028 efficiently and stably was finally obtained through multiple monoclonal screening. The cell line was further used to produce protein for in vivo anti-tumor activity studies in mice.

There have been no bispecific antibodies targeting TGF-β and PD-L1 on the market. M7824 from Merck is currently the most advanced and its clinical phase II results are very impressive. The variable region sequence of the PD-L1 portion of HB0028 and HB0029 in the present application is patent protected, and the GS linker and the TGF-βRII extracellular region portion can be publicly shared sequences, differing in that the receptor portion of HB0028 is located at the N-terminus of the monoclonal antibody and the receptor portion of HB0029 is located at the C-terminus of the monoclonal antibody, the latter having the same structure as that of Merck. It is shown from the results of the present application that, the expression and stability of HB0028 are better than those of HB0029 and the control drug 900544, and it can retain the binding activity of the TGF-βRII extracellular region better. Specifically, the in vitro activity of HB0028 is essentially comparable to that of M7824 from Merck. Moreover, from the in vivo results, HB0028 can achieve comparable clinical effects to that of the control drug M7824 by means of dose adjustment.

### Terms

To make the present application easier to understand, some technical and scientific terms are specifically defined below. Unless otherwise expressly defined herein, all other technical and scientific terms used herein have the meaning normally understood by one of ordinary skill in the art to which the present application belongs.

The Three-letter codes and One-letter codes of amino acids used in the present application are as described in J. biol. chem, 243, p 3558 (1968).

As used herein, the terms “administering/administration” and “treating/treatment” means the application of exogenous drugs, therapeutic agents, diagnostic agents or compositions to animals, humans, subjects, cells, tissues, organs or biofluids. “Administering/administration” and “treating” may refer to therapeutic, pharmacokinetic, diagnostic, research, and experimental methods. Treatment on cells may include the contact between reagents and cells, the contact between reagents and fluids, and the contact between fluids and cells. “Administering/administration” and “treating/treatment” also mean in vitro and ex vivo treatment with reagents, diagnostic and binding compositions or another kind of cells. “Treating/treatment”, when applied on humans, animals or study subjects, may refer to therapeutic treatment, prevention or preventive measures, research or diagnosis; and it may include the contact between anti-human PD-L1 antibodies and humans or animals, subjects, cells, tissues, physiological compartments or physiological fluids.

As used herein, the term “treating/treatment” means the administration of oral or topical therapeutic agents comprising any of the anti-PD-L1/TGF-β bifunctional antibodies of the present application and compositions thereof to a patient with one or more symptoms of a disease for which the therapeutic agents are known to have therapeutic effects. In general, the therapeutic agents may be administered to the patient in an amount effective to relieve one or more symptoms of the disease (therapeutically effective amount).

As used herein, the term “optional” or “optionally” means the subsequently described events or situations may but not necessarily occur. For example, “optionally including 1 to 3 antibody heavy chain variable regions” means that there may be but not necessarily antibody heavy chain variable regions of specific sequences, which may be 1, 2 or 3.

The “sequence identity” in the present application indicates the degree of identity between two nucleic acid or two amino acid sequences when optimally aligned and compared in the circumstances of appropriate mutations such as substitutions, insertions, or deletions. The sequence identity between sequences described in the present application and sequences to which they are identical can be at least 85%, 90% or 95%, and it can be at least 95%. Non-limiting examples may include 85%, 86%, 87%, 88%, 89%, 90%, 91%, 92%, 93%, 94%, 95%, 96%, 97%, 98%, 99%, 100%.

In general, “antibodies”, also known as “immunoglobulin”, can be natural or conventional antibodies in which two heavy chains are connected to each other via a disulfide bond and each heavy chain is connected to a light chain via a disulfide bond. There are two types of light chain, lamda (l) and kappa (k). There are five major classes of heavy chain (or isotypes), which determine the functional activity of antibody molecules: IgM, IgD, IgG, IgA, and IgE. Each chain contains a different sequence domain. A light chain can include two domains or regions: a variable domain (VL) and a constant domain (CL). A heavy chain can include four domains: a heavy chain variable region (VH) and three constant regions (CHL CH2 and CH3, collectively referred to as CH). The variable regions of both light chains (VL) and heavy chains (VH) determine the binding recognition and specificity for antigens. The constant domain (CL) of the light chain and the constant region (CH) of the heavy chain confer important biological properties such as antibody chain binding, secretion, transplacental mobility, complement binding and binding to Fc receptors (FcRs). The Fv fragment is the N-terminal portion of the Fab fragment of an immunoglobulin and is composed of the variable portions of one light chain and one heavy chain. The specificity of an antibody may depend on the structural complementarity between the antibody binding site and the epitope. The antibody binding site may consist of residues that are primarily derived from a hypervariable region or a complementarity determining region (CDR). Occasionally, residues from the non-hypervariable region or the framework region (FR) may affect the overall structure of the domain and thus affect the binding site. The complementarity determining region or CDR refers to an amino acid sequence that collectively defines the binding affinity and the specificity of the native Fv region of the native immunoglobulin binding site. The light chain and heavy chain of immunoglobulin can each have three CDRs, which can respectively be CDR1-L, CDR2-L, CDR3-L and CDR1-H, CDR2-H, CDR3-H. The antigen binding site of a conventional antibody can thus include six CDRs, including the set of CDRs from each of the heavy and light chain v-regions.

As used herein, the term “variable” indicates some portions of the variable region in an antibody differs in sequence, which contributes to the binding and specificity of various specific antibodies to their specific antigens. However, variability is not uniformly distributed throughout the variable region of the antibody. It is concentrated in three fragments in the light and heavy chain variable regions called either complementarity determining regions (CDRs) or hypervariable regions. The more conserved part of the variable region can be called the framework region (FR). The variable regions of native heavy and light chains each contains four FR regions, which are in a roughly β-folded configuration linked by three CDRs that form a linking loop, and in some cases can form a partially β-folded structure. The CDRs in each chain are held together closely through the FR region, and form the antigen-binding site of the antibody together with the CDRs of another chain (see Kabat et al., NIH Publ. No. 91-3242, Vol I, pages 647-669 (1991)). The constant regions may be not directly involved in the binding of antibodies to antigens, but they exhibit different effector functions, for example, they are involved in the antibody-dependent cytotoxicity of antibodies.

As used herein, the term “framework regions” (FRs) refers to the amino acid sequences interposed between the CDRs, i.e., those portions of the light and heavy chain variable regions of immunoglobulins that are relatively conserved among different immunoglobulins in a single species. The light chain and heavy chain of immunoglobulin can each have four FRs, which can respectively be referred to as FR1-L, FR2-L, FR3-L, FR4-L and FR1-H, FR2-H, FR3-H, FR4-H. Correspondingly, the light chain variable domain can thus be referred to as (FR1-L)-(CDR1-L)-(FR2-L)-(CDR2-L)-(FR3-L)-(CDR3-L)-(FR4-L), and the heavy chain variable domain can thus be represented as (FR1-H)-(CDR1-H)-(FR2-H)-(CDR2-H)-(FR3-H)-(CDR3-H)-(FR4-H). For example, the FRs of the present application can be human antibody FRs or derivatives thereof that are substantially identical to the naturally occurring human antibody FRs, that is, the sequence identity reaches 85%, 90%, 95%, 96%, 97%, 98% or 99%.

Upon acquiring the amino acid sequences of CDRs, those skilled in the art can easily determine the framework regions FR1-L, FR2-L, FR3-L, FR4-L and/or FR1-H, FR2-H, FR3-H, FR4-H.

As used herein, the term “human framework regions” refers to the framework regions that are substantially (about 85% or more, particularly 90%, 95%, 97%, 99% or 100%) identical to the naturally occurring human antibody framework regions.

As used herein, the term “monoclonal antibody” or “mAb” refers to an antibody molecule composed of a single amino acid against a particular antigen, and should not be construed as requiring any particular methods to produce the antibody. Monoclonal antibodies can be produced from a single clone of B cells or hybridomas, but can also be recombinant, i.e., produced by protein engineering.

As used herein, the term “antigen” or “target antigen” refers to a molecule or a portion thereof capable of being bound by an antibody or antibody-like binding protein. This term further refers to a molecule or a portion thereof that can be used in an animal to produce an antibody capable of binding to an epitope of the antigen. A target antigen can have one or more epitopes. For each target antigen recognized by an antibody or by an antibody-like binding protein, the antibody-like binding protein is able to compete with intact antibodies that recognize the target antigen.

As used herein, the term “affinity” is theoretically defined by the equilibrium association between an intact antibody and an antigen. The affinity of the dual antibody of the present application can be evaluated or determined by KD value (dissociation constant) (or by other determination means), such as Bio-layer interferometry (BLI), which can be measured and determined by using FortebioRed96 instrument.

As used herein, the term “adaptor” refers to one or more amino acid residues interposed in an immunoglobulin domain that provide sufficient mobility to the light and heavy chain domains to fold into an immunoglobulin with exchanged dual variable regions. For example, the adaptor element of the present application can be a GS connecting peptide, e.g., the amino acid sequence of the GS connecting peptide can be as set forth in SEQ ID NO: 3.

Anti-PD-L1 Antibody

Programmed cell death receptor-1 (PD-1) is a negative co-stimulatory molecule discovered in recent years which can be the CD28 immunoglobulin superfamily. PD-1 is commonly expressed in activated T cells, B cells and myeloid cells, and has two natural ligands, i.e., programmed death ligand 1 (PD-L1) and PD-L2, both belonging to the B7 superfamily, which are expressed in antigen-presenting cells, and PD-L1 is also expressed in a variety of tissues. Wherein, PD-L1 is an important negative immunomodulatory factor of PD-1, also known as B7-H1, the binding of which to PD-1 mediates the co-inhibitory signal of T-cell activation, inhibits T-cell activation and proliferation, and plays a negative regulatory effect similar to that of CTLA-4 to induce apoptosis of T cells. In addition, it has been shown in some research reports that tumor microenvironment can also protect tumor cells from destruction by immune cells, making them unrecognizable and undergo immune escape. Moreover, tumor microenvironment allows for persistent expression of PD-L1, making the immune function of tumor patients extremely decreased.

The laboratory of Chen Lieping, a Chinese scientist, first discovered that PD-L1 is highly expressed in tumor tissues and regulates the function of tumor-infiltrating CD8T cells. Therefore, immunomodulation targeting PD-1/PD-L1 is of great significance for anti-tumor. In recent years, clinical researches have been rapidly conducted on a variety of Anti-PD-1/PD-L1 antibodies in tumor immunotherapy. At present, Pembrolizumab and Nivolumab have been approved by FDA for use in the treatment of advanced melanoma, and recently, Nivolumab has also been approved by U.S.FDA for use in the treatment of advanced squamous non-small cell lung cancer. Additionally, MPDL3280A (an anti-PD-L1 monoclonal antibody), Avelumab (an anti-PD-L1 monoclonal antibody), etc. have also entered several late phase clinical studies covering non-small cell carcinoma, melanoma, bladder cancer and other tumor types.

In some embodiments, the heavy chain variable region (VH) of the anti-PD-L1 antibody can include the following three complementary determining regions (CDRs):
a CDR1 as set forth in SEQ ID NO: 12,
a CDR2 as set forth in SEQ ID NO: 13, and
a CDR3 as set forth in SEQ ID NO: 14; and/or
the light chain variable region (VL) of the anti-PD-L1 antibody can include the following three complementary determining regions (CDRs):
a CDR1′ as set forth in SEQ ID NO: 15,
a CDR2′ with an amino acid sequence of GIS, and
a CDR3′ as set forth in SEQ ID NO: 16.

The person skilled in the art may also modify or transform the anti-PD-L1 antibody of the present application by techniques well known in the art, such as adding, deleting and/or replacing one or several amino acid residues, thereby further increasing the affinity or structural stability of the anti-PD-L1, and obtain the results after modification or transformation by conventional assays.

TGF-β

TGF-β has a series of physiological functions including regulating the cell growth, differentiation, apoptosis, migration and infiltration, extracellular matrix formation, angiopoiesis, and immunomodulation, and plays an important role in embryonic development and individual maintenance of homeostasis. It is found in studies that, the embryos of TGF-β knockout mice fail to develop normally, resulting in the death of the mice. TGF-β can play different roles at different stages of tumor formation: at the early stage of tumor formation, activation of the TGF-β signaling pathway increases the expression of the cyclin-dependent kinase mechanism agents p15 and p21, leading to cell cycle arrest and apoptosis; at the late stage of tumor formation, tumor cells reverse the apoptosis-inducing effects of TGF-β through the following three pathways: 1) downregulation of the expression of p15 and p21 through the bypass pathway; 2) activation of the Ras/MAPK pathway; and 3) inactivating mutations of TGF-β receptors and downstream molecules. Thereafter, tumor cells secrete TGF-β in large quantities, which acts on the surrounding cells to promote stromal cell fibrosis, promote tumor angiogenesis, promote epidermal to mesenchymal cell transformation and cell transfer, thus inhibiting the activity of immune activating cells such as T cells, NK cells, dendritic cells, Th1 cells, M1 macrophages, etc. and promoting the production and activation of immune suppressive cells such as T regulatory cells, Th2 cells, M2 macrophages, etc., and ultimately promoting tumor development and metastasis (Hague S, Morris J C. Transforming growth factor-β: A therapeutic target for cancer[J]. Human Vaccines & Immunotherapeutics, 2017, 13 (8): 1741-1750.).

Due to the important role of TGF-β in tumor development, TGF-β and its signaling pathway-related molecules can be important therapeutic targets. Based on the different stages of the signaling pathway in which the target is located, therapeutic drugs can be divided into three categories: 1) TGF-β synthesis inhibitors; 2) TGF-β and receptor blockers; and 3) TGF-β downstream signaling pathway blockers. Antisense oligonucleotide is a kind of potent protein synthesis machinery agent. Trabederson AP12009 developed by Antisense Pharma Co. is an antisense oligonucleotide made up of 18 oligonucleotides targeting TGF-β mRNA which inhibits it from being translated into TGF-β protein. By being locally injected into the tumor site via a catheter, it can effectively inhibit tumor growth and prolong the survival of patients. Phase III clinical trials had been conducted on antisense oligonucleotides but were terminated in 2014 due to lack of enrolled patients. Monoclonal antibodies targeting TGF-β are the most mature researched TGF-β and receptor blockers, with the most advanced being Genzyme's GC1008 (clinical phase II) and CAT-192 (clinical phase VII), Novartis' NIS793 (clinical phase II), Boehringer Ingelheim and Eli Lilly's LY2382770 (clinical phase II) and Scholar Rock's GARP/TGF-β1 dual antibody SRK-181 (clinical phase I), and many other TGF-β monoclonal antibodies are in preclinical studies, making the competition very fierce. TGF-β receptor kinase inhibitors or the mechanism agents of downstream molecules ALK-5, such as LY2157299, LY2109761, SB-431542, etc., have all been shown to block TGF-β signaling pathway in animal models in vivo or in vitro, but the development on some drugs has been terminated due to drug resistance or poor in vivo pharmacokinetic properties. At present, only Eli Lilly's TGF-βRI small molecule inhibitor LY2157299 (Galunisertib) completed a phase III clinical trial (NCT02008318) in 2019. Soluble recombinant TGF-β receptor II or receptor III has been shown to be effective in inhibiting the growth of glioma, non-small cell lung cancer, breast cancer, and other tumors in mice, but the studies on them have not been put into clinical trials.

In one example of the present application, the anti-TGF-β-containing element in the bifunctional antibody can include an extracellular region of a TGF-β receptor.

In some embodiments, the TGF-β receptor can include TGF-βRI, and TGF-βRIII

In some embodiments, the anti-TGF-β element can include a TGF-βRII extracellular region, e.g., the amino acid sequence of the TGF-βRII extracellular region can be shown in SEQ ID NO: 2.

For example, regardless of which end of the anti-PD-L1 antibody the TGF-βRII extracellular region of the present application is attached to, two identical TGF-βRII extracellular regions are connected by an adaptor and thus appear as a dimer.

Bifunctional Antibody (Bispecific Antibody)

Bispecific Antibody (bsAb) is a non-natural antibody that can simultaneously target two different antigens or proteins, block two different signaling pathways, and stimulate specific immune responses. Its specificity and biofunctionality are increasingly important in the immunotherapy of tumors, and it has become a hot spot for research in antibody engineering for the treatment of tumors in the world today. Studies show that bispecific antibodies in the immunotherapy of tumors mainly have the advantages of mediating the killing of tumors by immune cells; binding dual targets and blocking dual signaling pathways to play unique or overlapping functions, which can effectively prevent drug resistance; having strong specificity and targeting ability and reducing off-target toxicity; effectively reducing the cost of treatment, etc. Therefore, the use of bispecific antibody drugs can reduce the chance of tumor cell escape, remove tumor cells and improve the efficacy.

Bispecific antibodies can be prepared by means of double hybridoma cells, chemical coupling, and recombinant genes, where the recombinant gene technique is flexible in terms of binding sites and yield. According to incomplete statistics, there are more than 60 types of bispecific antibodies currently. According to their characteristics and structural differences, there are two main types of bispecific antibody structures: bispecific antibodies containing Fc fragments (IgG-like bispecific antibodies with Fc-mediated effector functions) and bispecific antibodies without Fc fragments (non-IgG-like bispecific antibodies, which act through antigen binding and have the advantages of small molecular weight and low immunogenicity). Amgen's bispecific antibody Blincyto (Blinatumomab) was approved for marketing by the U.S. FDA on Dec. 3, 2014, for the treatment of acute lymphocytic leukemia. Blinatumomab can be a CD19 and CD3 bispecific antibody, and Blincyto (Blinatumomab) is the first bispecific antibody approved by the U.S. FDA.

As used herein, the terms “bispecific antibody”, “bifunctional antibody”, “the antibody of the present application”, “the dual antibody of the present application”, “dual antibody”, and “bifunctional fusion antibody” are used interchangeably and refer to anti-PD-L1/TGF-β bispecific antibody that binds both PD-L1 and TGF-β.

In the present application, the bifunctional antibody can include:
(a) an anti-PD-L1 antibody or element; and
(b) an anti-TGF-β antibody or element connected to the anti-PD-L1 antibody or element.

In some embodiments, the bifunctional antibody has a structure represented by formula Ia or Ib from N-terminus to C-terminus:

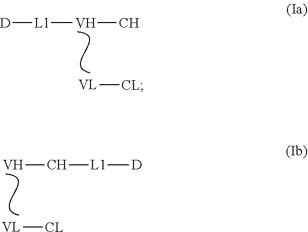

wherein,
“-” represents a peptide bond;
“ [symbol] ” represents a disulfide bond;
D can be an anti-TGF-β element;
L1 can be none or an adaptor element;
VH represents the heavy chain variable region of the anti-PD-L1 antibody;
CH represents the heavy chain constant region of the anti-PD-L1 antibody;
VL represents the light chain variable region of the anti-PD-L1 antibody;
CL represents the light chain constant region of the anti-PD-L1 antibody;
wherein, the bifunctional antibody can have an activity of simultaneously binding to PD-L1 and TGF-β.

In formula Ia or Ib, for example, the H chain can be as set forth in SEQ ID NO: 1, and the L chain can be as set forth in SEQ ID NO: 7.

And two sequences as shown in the structural formula Ia or Ib can be connected through the disulfide bond of the H chain to form a symmetrical bifunctional antibody structure.

The dual antibody of the present application may include not only intact antibodies, but also fragments of immunologically active antibodies or fusion proteins formed by antibodies with other sequences. Therefore, the present application can further include fragments, derivatives or anologues of the antibodies. As used herein, the terms “fragments”, “derivatives” and “anologues” refer to polypeptides that retain substantially the same biological functions or activities as those of the antibody of the present application. The polypeptide fragments, derivatives or anologues of the present application can be: (i) polypeptides with one or more conserved or non-conserved amino acid residues (maybe conserved amino acid residues) substituted, while such substituted amino acid residues may or may not be encoded by genetic codes; or (ii) polypeptides with substituent groups in one or more amino acid residues; or (iii) polypeptides formed by fusing a mature polypeptide to another compound (such as, a compound that extends the half-life of the polypeptide, e.g., polyethylene glycol); or (iv) polypeptides formed by fusing an additional amino acid sequence to this polypeptide sequence (e.g., a leader sequence, or a secretory sequence, or a sequence or a proteinogen sequence for purifying the polypeptide, or a fusion protein formed with a 6His-tag). According to the teachings herein, these fragments, derivatives and anologues all fall within the scope well known to those skilled in the art.

The dual antibody of the present application refers to antibodies with anti-PD-L1 and anti-TGF-β activity, which may include two structures of formula I above. This term can also include variant forms of antibodies that have the same function as the dual antibody of the present application and may include two structures of formula I above. These variant forms can include, but not limited to, deletion, insertion and/or substitution of one or more (generally can be 1-50, e.g., 1-30, 1-20, or 1-10) amino acids, as well as addition of one or more (generally can be less than 20, e.g., less than 10, or less than 5) amino acids at the C-terminus and/or the N-terminus. For example, in this art, substitution with amino acids with close or similar properties does not usually change the function of the protein. Further for example, addition of one or more amino acids at the C-terminus and/or the N-terminus does not usually change the function of the protein, either. This term can also include active fragments and active derivatives of the dual antibody of the present application.

The variant forms of the dual antibody can include: homologous sequences, conserved variants, allelic variants, natural mutants, inducible mutants, proteins encoded by the DNA that can hybridize with the DNA encoding the antibody of the present application under high or low stringency conditions, as well as peptides or proteins obtained using antisera against the antibody of the present application.

In the present application, “conservative variants of the dual antibody of the present application” refer to polypeptides formed by the substitution of up to 10, e.g. up to 8, e.g. up to 5, e.g. up to 3 amino acids with amino acids of similar or close properties compared to the amino acid sequence of the dual antibody of the present application. These conservative variant polypeptides are preferably produced by amino acid substitutions according to Table A.

TABLE A

| Initial residue | Representative substitutions | Substitution example |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

Coding Nucleic Acids and Expression Vectors

The present application also provides polynucleotide molecules encoding the antibodies above or fragments thereof or fusion proteins thereof. The polynucleotides of the present application can be in a form of DNA or RNA. The forms of DNA can include cDNA, genomic DNA, or artificially synthesized DNA. DNA can be single-stranded or double-stranded. DNA can be a coding chain or a non-coding chain. The polynucleotides encoding the mature polypeptides of the present application can include: coding sequences only encoding mature polypeptides; coding sequences and various additional coding sequences of mature polypeptides; and coding sequences (and optionally additional coding sequences) and non-coding sequences of mature polypeptides.

The term “a polynucleotide encoding a polypeptide” can be a polynucleotide that encodes the polypeptide, and can also be a polynucleotide that can further include an additional coding and/or non-coding sequence.

The nucleic acid (and nucleic acid combination) of the present application can be used to produce the recombinant antibodies of the present application in a suitable expression system.

The present application also relates to a polynucleotide that hybridizes to the sequence above and has at least 50%, for example at least 70%, for example at least 80% identity between the two sequences. The present application particularly relates to a polynucleotide which is hybridizable to the polynucleotide described herein under stringent conditions. In the present application, “stringent conditions” refer to: (1) hybridization and elution at lower ionic strength and higher temperature, such as 0.2×SSC, 0.1% SDS, 60° C.; or (2) addition of denaturant during the hybridization, such as 50% (v/v) formamide, 0.1% fetal bovine serum/0.1% Ficoll, 42° C., etc.; or (3) hybridization occurs only when the identity between the two sequences is at least 90%, e.g. it can be above 95%. In addition, polypeptides encoded by hybridizable polynucleotides have the same biological function and activity as the mature polypeptides.

The full-length nucleotide sequence of the antibody of the present application or the fragment thereof can usually be obtained by means of PCR amplification, recombination or artificial synthesis. A feasible approach is to synthesize the sequences in question by artificial synthesis, especially when the fragments are short. Generally, fragments with very long sequences can be obtained by synthesizing multiple small fragments and then ligating them. In addition, the coding sequence of a heavy chain can also be fused to an expression tag (e.g., 6His) to form a fusion protein.

Once the relevant sequences have been obtained, the recombination method can be used to obtain the sequences in question in large quantities. The sequences in question are usually obtained by cloning them into vectors, then transferring into cells, and then isolating from proliferated host cells by conventional methods. The biological molecules (nucleic acids, proteins, etc.) covered by the present application can include biological molecules in isolated forms.

At present, it has been possible to obtain the DNA sequences encoding the proteins (or fragments thereof, or derivatives thereof) of the present application completely through chemical synthesis. After then, the DNA sequences can be introduced into a variety of existing DNA molecules (or, such as vectors) and cells known in the art. In addition, mutations can also be introduced into the protein sequence of the present application through chemical synthesis.

The present application further relates to vectors containing the above appropriate DNA sequences and appropriate promoters or control sequences. These vectors can be used to transform appropriate host cells to enable them to express proteins.

Host cells can be prokaryotic cells, e.g., bacterial cells; or can be lower eukaryotic cells, e.g., yeast cells; or can be higher eukaryotic cells, e.g., mammalian cells. Representative examples include: E. coli, Streptomyces sp.; bacterial cells of Salmonella typhimurium; fungal cells, which can be, e.g., yeast; insect cells of Drosophila S2 or Sf9; animal cells such as CHO, COST, 293 cells, etc.

Transformation of host cells with recombinant DNA can be performed by conventional techniques well known to those skilled in the art. When the host can be a prokaryote such as E. coli, competent cells capable of absorbing DNA can be harvested after an exponential growth period and treated with CaCl₂, using procedures well known in this field. Another method is using MgCl₂. If desired, the transformation can also be performed by electroporation. When the host is a eukaryote, the following DNA transfection methods can be used: calcium phosphate co-precipitation, conventional mechanical methods such as micro-injection and electroporation, and liposome packaging, etc.

The obtained transformants can be cultured by conventional methods to express the polypeptides encoded by the genes of the present application. Depending on the host cells used, the medium used in the culture can be selected from various conventional media. The culture is conducted under conditions suitable for the growth of host cells. When the host cells have grown to an appropriate cell density, the selected promoters are induced using a suitable method (e.g., temperature shift or chemical induction), and the cells are further cultured for a period of time.

Under the early culture conditions, the expression of bispecific antibodies can reach 3.9 g/L, and the purity can be all above 97%, and lactic acid can be well metabolized during the culture process.

The recombinant polypeptides in the above methods can be expressed within the cells or on the cell membrane, or secreted outside the cells. If desired, the recombinant proteins can be isolated and purified by a variety of isolation methods based on their physical, chemical and other properties. These methods are well known to those skilled in the art. Examples of these methods can include, but not limited to, conventional renaturation treatment, treatment with protein precipitant (salt precipitation), centrifugation, cell disruption by osmosis, ultrasonication, ultracentrifugation, molecular sieve chromatography (gel filtration), adsorption chromatography, ion exchange chromatography, high performance liquid chromatography (HPLC) and combinations of these methods with other various liquid chromatography techniques.

The dual antibody of the present application can be used alone, and can also be used in combination or conjugating with a detectable marker (which can be for diagnostic purpose), a therapeutic agent, or a combination of any of the above substances.

The detectable markers for diagnostic purpose can include, but not limited to, fluorescent or luminescent markers, radioactive markers, MRI (Magnetic resonance imaging) or CT (computed tomography) contrast media, or enzymes capable of producing detectable products.

The therapeutic agents that can be combined or conjugated with the antibody of the present application can include, but not limited to, 1. radionuclides; 2. biotoxins; 3. cytokines, such as IL-2, etc.; 4. gold nanoparticles/nanorods; 5. virions, 6. lipidosomes; 7. nanomagnetic particles, 8. tumor therapeutic agents (e.g., cisplatin) or any form of anti-tumor drugs.

Pharmaceutical Composition

The present application further provides a composition. For example, the composition can be a pharmaceutical composition containing the bispecific antibody of the present application or an active fragment thereof or a fusion protein thereof, and a pharmaceutically acceptable carrier. Generally, these substances can be formulated in a non-toxic, inert and pharmaceutically acceptable aqueous carrier medium, of which the pH can generally be about 5-8, for example, the pH can be about 6-8, although the pH may vary with the nature of the substance being formulated and the condition to be treated. The formulated pharmaceutical composition can be administered through conventional routes, including, but not limited to: intravenous injection, intravenous infusion, subcutaneous injection, local injection, intramuscular injection, intratumoral injection, intra-abdominal injection (e.g., intraperitoneal), intracranial injection, or intracavitary injection.

The pharmaceutical composition of the present application can be directly used to bind PD-L1 and/or TGF-β, and thus can be used for the treatment of tumors. In addition, other therapeutic agents can also be used concurrently.

The pharmaceutical composition of the present application can contain a safe and effective amount (for example, 0.001-99 wt %, e.g., it may be 0.01-90 wt %, e.g., it may be 0.1-80 wt %) of the above nanoantibody (or a conjugate thereof) of the present application and a pharmaceutically acceptable carrier or excipient. Such carriers can include, but not limited to, brine, buffer, glucose, water, glycerin, ethanol, and combinations thereof. The pharmaceutical formulation should be matched to the mode of administration. The pharmaceutical composition of the present application can be made in the form of injections, for example, it can be prepared by conventional methods with physiological saline or an aqueous solution containing glucose and other adjuvants. The pharmaceutical compositions, such as injections and solutions, should be manufactured under sterile conditions. The active ingredient is administered at a therapeutically effective amount, e.g. about 10 μg/kg body weight to about 50 mg/kg body weight per day. In addition, the polypeptide of the present application can also be used together with other therapeutic agents.

In the present application, the bispecific antibody can be used alone to get the optimal desired response by adjusting the dosing regimen. For example, a single dose, multiple doses over a period of time, or the dose can be proportionally reduced or increased according to the urgency of the treatment situation.

When the pharmaceutical composition is used, the immunoconjugate can be administered to a mammal at a safe and effective amount which is usually at least about 10 μg/kg body weight, and in most instances, not more than about 50 mg/kg body weight, for example, the dose can be about 10 μg/kg body weight to about 10 mg/kg body weight. Of course, the specific dose should also take into account the route of administration, the health status of the patient and other factors, which are all within the skill of the skilled physicians.

The Main Advantages of the Present Application Include:
(a) the bifunctional antibody of the present application can simultaneously bind to PD-L1 and TGF-β, restore the activation of T cells, and inhibit TGF-β/SMAD signaling pathway.
(b) the bifunctional antibody HB0028 of the present application has a very good structural stability, and can retain the binding activity of the TGF-βRII extracellular region better.
(c) the bifunctional antibody HB0028 of the present application can be expressed efficiently and stably in CHO host cells, and is easy to produce.

The present application is further described below in connection with specific embodiments. It should be understood that these embodiments are intended to illustrate the present application only and are not intended to limit the scope of the present application.

Experimental methods for which no specific conditions are indicated in the following embodiments are generally conducted following conventional conditions, such as those described in Sambrook et al., Molecular Cloning: A Laboratory Manual (New York. Cold Spring Harbor Laboratory Press, 1989), or as recommended by the manufacturer. Unless otherwise indicated, percentages and parts are percentages by weight and parts by weight.

### EXAMPLES

### Example 1 Construction of Expression Vectors

Suzhou GENEWIZ Biological Technology Co, Ltd. (GENEWIZ for short) was entrusted to synthesize N-fusion and C-fusion genes with amino acids 24-159 (ECD₂₄₋₁₅₉) in the human TGF-βRII extracellular region (Accession No.: P37173), wherein N-fusion and C-fusion indicate N-terminal and C-terminal fusion of TGF-βRII ECD with the heavy chain of a humanized PD-L1 antibody through a GS flexible linker, respectively. For gene synthesis, the HindIII endonuclease recognition site was added at the 5′ end of N-fusion, the heavy chain variable region of the PD-L1 antibody (HB0023) and part of the C_{H}1 gene sequence were attached downstream of the receptor ECD, and the NheI endonuclease recognition site was added at the 3′ end. The 5′ end of C-fusion initiates from the SexAI endonuclease recognition site of C_{H}3 in the heavy chain constant region of the PD-L1 antibody (HB0023) and includes part of C_{H}3 and receptor ECD genes with the XmaI endonuclease recognition site added at its 3′ end. The synthesized gene was constructed into the pUC57 vector by GENEWIZ to prepare a mini-scale recombinant plasmid DNA and puncturing bacteria containing the recombinant plasmid, and the puncturing bacteria can be used to amplify and prepare more plasmids for later use. The prepared N-fusion plasmids and the heavy chain expression vectors (Huabo Code: 400078) of the PD-L1 antibody were respectively double digested with HindIII and NheI, and purified. After then, the fragments and vectors were ligated with T4 ligase, and the L234A/L235A (EU numbering rule) mutation on the C_{H}2 domain of the backbone human IgG1 used in 400078 was replaced with wild-type human IgG1, to construct the resulting expression vector as the HB0028 heavy chain expression vector with PD-L1 and TGF-β bispecific antibodies fused at the N-terminus, with its number being 500054. For construction of the heavy chain expression vector of the C-fusion bispecific antibody HB0029, a plasmid containing the C-fusion gene provided by GENEWIZ was used as the template, and the target gene fragment was amplified by PCR using primers (upstream: AGGAGATGACCAAGAACCAGGTAAGTTTGACCTGCCT (SEQ ID NO: 10), downstream: ACCGCGAGAGCCCGGGGAGCGGGGGCTTGCCGGCCGTCGCA (SEQ ID NO: 11), synthesized by GENEWIZ). After the PD-L1 heavy chain expression vector 400078 was double digested with SexAI and XmaI, the PCR product was ligated with an enzyme digestion vector by using an In-fusion recombinase (Takara, Item No. 639650). Similarly, the L234A/L235A mutation on the C_{H}2 domain of the backbone human IgG1 used in 400078 was replaced with wild-type human IgG1, to construct the resulting HB0029 heavy chain expression vector with PD-L1 and TGF-β bispecific antibodies fused at the C-terminus, with its number being 500055. The light chain of the bispecific antibody is the same as that of the parent PD-L1 humanized antibody, with its number being 400085. The sequence of the bispecific antibody is as below:
the amino acid sequence of HB0028 heavy chain 500054 is:

(SEQ ID NO: 1)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSGGGGSGQVQLVQSGAEVKKPGASVKVSCKASGYAFTGYTIHWVRQ
APGQRLEWMGWFYPGSGTLKYSEKFQGRVTITRDKSLSTAYMELSSLRS
EDTAVYYCARHGTGTLMAMDYWGQGTLVTVSSASTKGPSVFPLAPSSKS
TSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVM
HEALHNHYTQKSLSLSPG

wherein, TGF-βRII ECD₂₄₋₁₅₉ is:

(SEQ ID NO: 2)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD

the GS linker is:

(SEQ ID NO: 3)
GGGGSGGGGSGGGGSGGGGSG

the sequence of the heavy chain variable region of the PD-L1 antibody (the underlined parts are CDR regions, which are classified on basis of the IMGT system) is:

(SEQ ID NO: 4)
QVQLVQSGAEVKKPGASVKVSCKASGYAFTGYTIHWVRQAPGQRLEWMG
WFYPGSGTLKYSEKFQGRVTITRDKSLSTAYMELSSLRSEDTAVYYCAR
HGTGTLMAMDYWGQGTLVTVSS

the sequence of the heavy chain constant region of the antibody is:

(SEQ ID NO: 5)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPG

the amino acid sequence of the HB0029 heavy chain 500055 (of which the sequences of the antibody variable region, the constant region, the linker and TGF-βRII are the same as those of HB0028, not separately listed here):

(SEQ ID NO: 6)
QVQLVQSGAEVKKPGASVKVSCKASGYAFTGYTIHWVRQAPGQRLEWMG
WFYPGSGTLKYSEKFQGRVTITRDKSLSTAYMELSSLRSEDTAVYYCAR
HGTGTLMAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGGGGGSGGGGSGGGGSGGGGSGIPPHVQKSVNNDMIVTDNNG
AVKFPQLCKFCDVRFSTCDNQKSCMSNCSITSICEKPQEVCVAVWRKND
ENITLETVCHDPKLPYHDFILEDAASPKCIMKEKKKPGETFFMCSCSSD
ECNDNIIFSEEYNTSNPD

the amino acid sequence of the light chain 400085 is:

(SEQ ID NO: 7)
DVVMTQTPLSLSVTPGQPASISCKSSQSLANSYGNTYLSWYLHKPGQSP
QLLIYGISNRFSGVPDRFSGSGSGTDFTLKISRVEAEDVGVYYCLQGTH
QPPTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

wherein, the sequence of the light chain variable region (the underlined parts are CDR regions, which are classified on basis of the IMGT system) is:

(SEQ ID NO: 8)
DVVMTQTPLSLSVTPGQPASISCKSSQSLANSYGNTYLSWYLHKPGQSP
QLLIYGISNRFSGVPDRFSGSGSGTDFTLKISRVEAEDVGVYYCLQGTH
QPPTFGQGTKLEIK

the sequence of the light chain constant region of the antibody is:

(SEQ ID NO: 9)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

### Example 2 Expression and Purification of Fusion Proteins

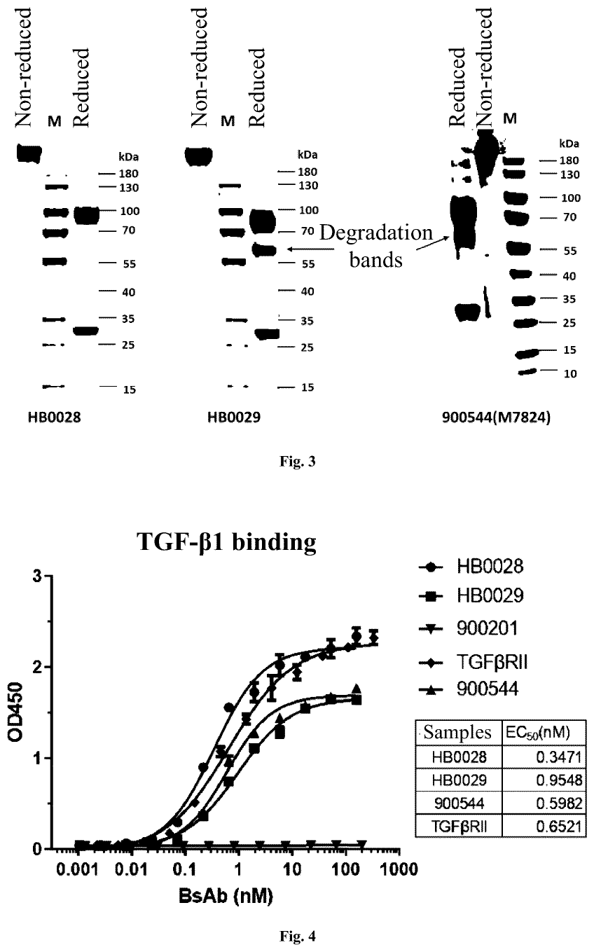

The expression of protein in the present application is divided into transient transfection expression and stable transfection expression. For transient transfection expression, the constructed heavy chain expression vectors 500054 and 500055 were respectively mixed with the light chain vector 400085 at a ratio of 1:1, pre-incubated with PEI (Polyetherimide), and then co-transfected into CHO-S (Thermo Fisher Scientific, R80007) cells, and cultured for 7 days at 32° C., 5% CO₂ and 125 rpm/min. The supernatant was then collected by centrifugation and purified for later use. For stable transfection expression, the constructed heavy chain expression vectors 500054 and 500055 were respectively mixed with the light chain vector 400085 at a ratio of 1:2 and added into blank CHO-K1 cells, mixed with a culture medium, and transfected by electroporation at a pulse voltage of 250 to 300 V. The stably transfected cell clones were screened with MSX under pressurization, and the monoclonal cell lines stably and efficiently transfected with HB0028 and HB0029 antibodies were screened by the limiting dilution method. After expanding the suspension culture and adding the feed required for the cell growth, the supernatant was collected by centrifugation after about 14 days. In order to compare with the M7824 control drug from Merck, Germany, the inventor synthesized the target gene based on the M7824 gene sequence published in the patent and loaded it into an expression vector, which was expressed and purified using the same transient transfection expression method. The collected supernatant was filtered through a 0.45 μm filter membrane, and the filtrate was collected. After purifying the filtrate over a Protein A affinity column, target proteins were obtained, in which M7824 was numbered as 900544. The purified target proteins were detected by SEC_UPLC for purity. The results demonstrated that the purity of HB0028 was higher than 95%, and the purity of HB0029 and 900544 was lower with obvious degradation bands. The target protein bands in reduced and non-reduced states were detected by SDS-PAGE, with the results shown in FIG. 3. The above results demonstrated that, the expression and stability of HB0028 were superior to those of HB0029 and the control drug 900544.

### Example 3 Binding Activity of Fusion Protein to Targets

3.1 Detection on the Binding Activity of Fusion Protein to Human TGF-β by the ELISA Method

TGF-β1 (ACRO, TG1-H4212) or TGF-β3 (R&D, 8420-B3-025) was diluted with PBS to 0.5 μg/ml, added into 96-well ELISA plates at 100 μl/well, coated at 4° C. overnight, and blocked with blocking buffer for 1 h after washing the plates with PB ST. Starting from 30 μg/ml, the samples to be tested were diluted in 3-fold gradient for 12 gradients, with TGF-βRII-Fc (ACRO, TG2-H5252) and the control drug 900544 (genes synthesized based on the sequence of the PD-L1/TGF-β dual antibody M7824 in the patent to Merck and expressed independently by Huabo Biotech) as the positive control and 900201 (which is a non-target antigen-targeted human IgG1 isotype control antibody used for multiple tests and as the negative control) as the negative control, added at 100 μl/well and reacted at room temperature for 2 h. After washing with PBST, into the plates was added HRP-labeled anti-human IgG secondary antibody (1:5000 dilution) at 100 μl/well, reacted at room temperature for 30 min, and then washed with PBST. After developing with TMB color development solution for 5 min, the reaction was terminated with sulfuric acid, and OD450 value was read by a microplate reader.

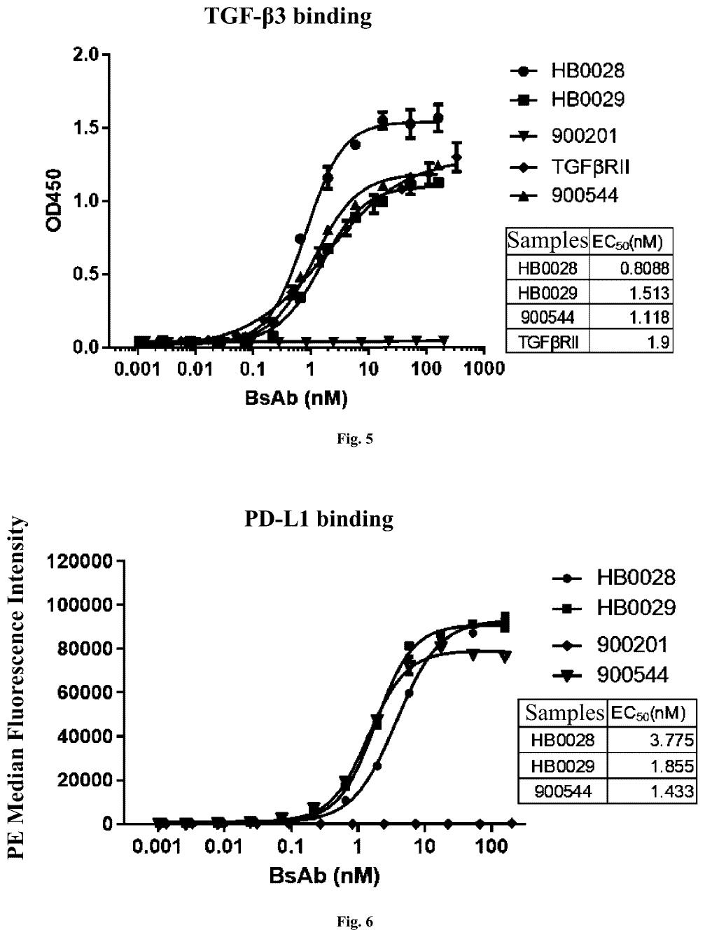

The results as shown in FIGS. 4 and 5 demonstrated that both HB0028 and HB0029 can effectively bind free TGF-β protein, and the binding activity of HB0028 is stronger than that of HB0029 and the control drug 900544.

3.2 Detection on the Binding Activity of Fusion Protein to Human PD-L1 by the FACS Method

CHO-K1 cells overexpressing human PD-L1 were taken and resuspended to 1×10⁶/ml and added into a 96-well plate at 20 μl/well. Starting from 30 μg/ml, the samples to be tested were diluted in 3-fold gradient for 12 gradients, with 900201 as the negative control and 900544 as the positive control antibody, added at 20 μl/well, incubated at room temperature for 30 min, and washed twice by centrifugation with 1% BSA-PBS. Into each well was added 20 μl of PE fluorescence labeled anti-human IgG secondary antibody (Jackson Immunoresearch, 109-115-098), incubated at room temperature for 15 min, washed three times by centrifugation, and then tested for the emission intensity at 580 nm by a flow cytometer Canto II (BD), with the results indicated by median fluorescence intensity (MFI).

The results as shown in FIG. 6 demonstrated that both HB0028 and HB0029 can effectively bind the human PD-L1 target protein on the cell membrane, and the binding activities of the samples to the antigen PD-L1 on the cell surface are comparable.

3.3 Detection on the Binding Activity of Fusion Protein to Dual Targets of PD-L1 and TGF-β by the FACS Method

The serially-diluted samples to be tested were pre-mixed with 3 μg/ml of TGF-β1 protein and incubated for 30 min with 900201 as the negative control and 900544 as the positive control antibody. After then, CHO-K1 cells overexpressing human PD-L1 were taken and resuspended to 1×10⁶/ml, added into a 96-well plate at 20 μl/well, and incubated for min after being mixed uniformly. The plate was washed twice by centrifugation with 1% BSA-PBS. Into each well was added 20 μl of PE fluorescence labeled anti-human TGF-β1 secondary antibody (1:100), incubated at room temperature for 15 min, washed three times by centrifugation, and then tested for the emission intensity at 580 nm by a flow cytometer Canto II (BD).

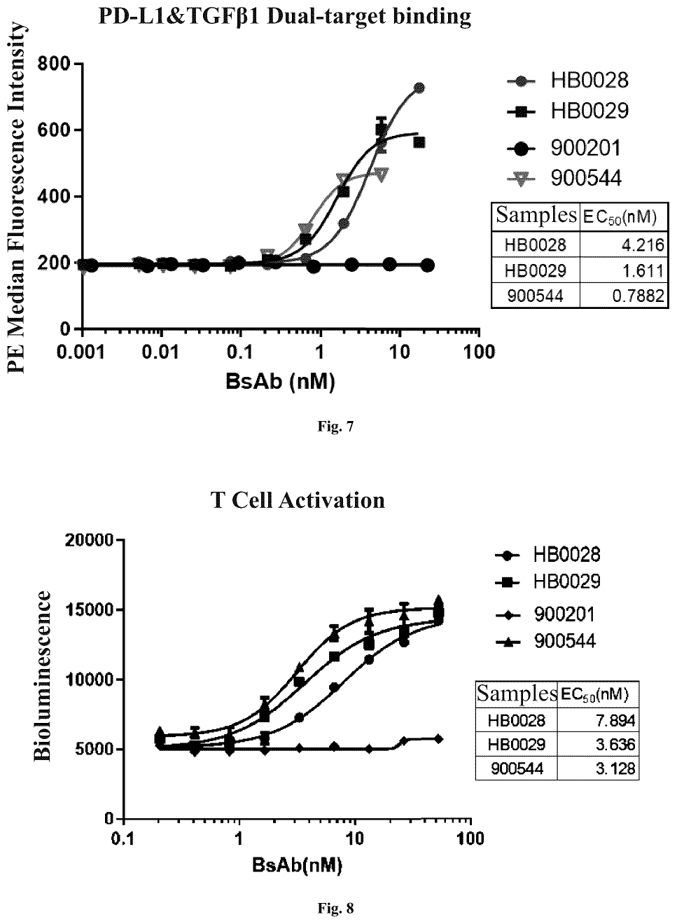

The results as shown in FIG. 7 demonstrated that the fusion protein can effectively bind both human PD-L1 on the cell membrane and free TGF-β target protein. Although at the same concentration, the binding strength of HB0028 to the dual targets is weaker than that of HB0029 and the control drug 900544, the platform above curve of the HB0028 molecules is the highest at the saturation concentration, that is, it can bind the dual targets more efficiently.

### Example 4 Detection of Biological Activity of Fusion Protein In Vitro by a Reporter Gene Method

4.1 Bifunctional Antibody Blocks PD-L1 to Restore T Cell Activation

The assay system consists of two genetically engineered cell lines: Jurkat-NFAT-PD-1-5B8 cells (PD-1 effector cells) which are Jurkat T cells stably expressing human PD-1 and NFAT-inducible luciferase; and CHO-K1-OS8-PD-L1-8D6 cells (PD-L1 target cells) capable of stably expressing human PD-L1 and TCR-activating antibody the OKT3 single-chain antibody on the cell surface. When the two types of cells are co-cultured, PD-1/PD-L1 interaction inhibits TCR signaling and NFAT-mediated luciferase activity. The addition of an antibody that can block either PD-1 or PD-L1 can relieve the inhibitory signal, thereby restoring the activation of the TCR signaling pathway and enhancing the NFAT-mediated luciferase activity.

The antibodies to be tested were diluted to 30000 ng/ml with culture medium, and then diluted in 2-fold gradient for 8 concentrations, for a total of 9 concentration gradients. Target cells Jurkat-NFAT-PD-1-5B8 were counted and resuspended at 5×10⁵/ml, then plated into a 96-well white-bottom plate at 30 μl per well; effector cells CHO-K1-OS8-PD-L1-8D6 were counted and resuspended at 5×10⁵/ml, then plated at 30 μl per well; the diluted samples to be tested were added at 30 μl per well. After mixing uniformly, they were incubated in a CO₂ incubator at 37° C. for 6 hours. The final working concentrations of the antibodies were tested to be 10000 ng/ml, 5000 ng/ml, 2500 ng/ml, 1250 ng/ml, 625 ng/ml, 312.5 ng/ml, 156.25 ng/ml, 78.125 ng/ml, and 39.063 ng/ml. At the end of incubation, the culture plate was equilibrated at room temperature for at least 15 min, and then the equilibrated Bio-Glo™ Luciferase Assay substrate buffer solution was added to a 96-well white plate at 90 μl/well, and reacted at room temperature for 20 min in dark. Full wavelength readings were taken using the MD SpectraMax® i3x microplate reader. Data were analyzed on GraphPad Prism 8 software by fitting a four-parameter equation with RLU values vs. antibody working concentrations.

The results as shown in FIG. 8 demonstrated that the bifunctional antibodies HB0028 and HB0029 can effectively restore the activation of T cells, and the abilities of the samples to activate T cells in vitro are comparable.

4.2 Inhibition of Bifunctional Antibody on TGF-β

After TGF-β ligand bound to the type II receptor on the cell membrane, the type II receptor recruited and phosphorylated the type I receptor, which in turn phosphorylated the receptor-regulated SMAD2/SMAD3 proteins, both of which bound to the SMAD4 protein, and the final complex entered the nucleus and was involved in regulating the expression of the target gene. The mouse breast cancer cells 4T1 were transfected with Cignal Lenti SMAD Reporter (luc) (QIAGEN, CLS-017L) reporter gene expression vectors, and the stably expressed cell lines were then screened with antibiotics, which were named as 4T1-SMAD cells and can be used to detect the activation of TGF-β and the blocking effect of antibodies.

4T1-SMAD cells were collected and resuspended at 5×10⁵/ml, and then plated into a 96-well white-bottom plate at 100 μl per well. The antibodies to be tested, the positive control 900544 and the negative control 900201 were diluted with a culture medium to 500 ng/ml, then diluted in 1.5-fold gradient for 8 gradients to 20000 ng/ml with TGF-βRII-Fc (ACRO, TG2-H5252) as the positive control, and further diluted in 3-fold gradient for 8 gradients. The diluted antibodies were added into a 96-well plate at 50 μl/well, incubated for 2 h, and further incubated with 501A1 of 20 ng/ml diluted TGF-β1 (ACRO, TG1-H4212) overnight. The cell culture supernatant was removed by centrifugation, then 30 μl of Bio-Glo™ Luciferase Assay substrate buffer solution (Promega, G7940) was added and reacted at room temperature for 5 min in dark. Full wavelength readings were taken using the MD SpectraMax® i3x microplate reader. Data were analyzed on GraphPad Prism 8 software by fitting a four-parameter equation with RLU values vs. antibody working concentrations.

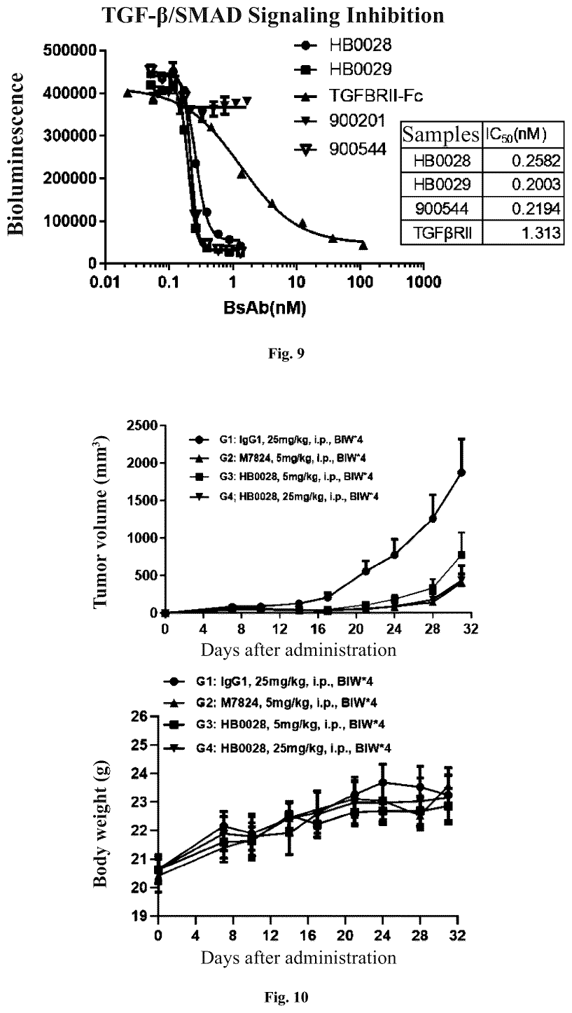

The results as shown in FIG. 9 demonstrated that the bifunctional antibodies HB0028 and HB0029 can effectively inhibit the transduction of TGF-β/SMAD signaling pathway, and the inhibitory activities of the samples were very close.

### Example 5 Detection of the Affinity Between 11B0028 and its Target Species Using BIAcore

For detecting the affinity of HB0028 for different species of PD-L1 antigens, a coupled Anti-human IgG (Fc) chip was used to capture HB0028 samples as the ligands and different species of PD-L1 antigens as analytes for multi-power cycling kinetic assay. For detecting the affinity of HB0028 for different species of—TGF-β antigens, a Protein A chip was used to capture HB0028 samples as the ligands and different species of TGF-β proteins as analytes for multi-power cycle kinetic assay. Flow rate: 30 μl/min, binding: 120 s, dissociation: 600 s, analyzing the kinetic constants by Fit local using a 1:1 binding mode.

The results were shown in Table 1. According to the results of the multi-power cycling assay, it can be known that the HB0028 antibody does not bind to PD-L1 in mice, rats and rabbits, while the KD values of affinity for PD-L1 in monkeys and humans are nM and 2.45 nM, respectively. At the TGF-β receptor end, HB0028 has an affinity of 10⁻¹¹ M for human, mouse/rat TGF-β1 and human TGF-β3, while this molecule does not bind to the precursor of TGF-β1 (Human LAP, Mouse Latent TGF-β1). Compared with the high affinity for TGF-β1 and TGF-β3, HB0028 has an affinity of 10-09 M for TGF-β2, and there is no difference among various species.

TABLE 1
Affinity between HB0028 and its targets of different species

| Analytes | ka (1/Ms) | kd (1/s) | KD (M) |
|---|---|---|---|
| Mouse PD-L1 | NB | NB | NB |
| Rat PD-L1 | NB | NB | NB |
| Rabbit PD-L1 | NB | NB | NB |
| Monkey PD-L1 | 2.17E+05 | 1.28E−03 | 5.87E−09 |
| Human PD-L1 | 1.90E+05 | 4.65E−04 | 2.45E−09 |
| Human TGF-β1 | 4.83E+07 | 5.50E−04 | 1.14E−11 |
| Mouse/Rat TGF-β1 | 9.05E+05 | 2.49E−06 | 2.75E−12 |
| Human LAP | NB | NB | NB |
| Mouse precursor TGF-β1 | NB | NB | NB |
| Human TGF-β2 | 5.02E+07 | 7.84E−02 | 1.56E−09 |
| Mouse/Rat TGF-β2 | 2.33E+07 | 4.60E−02 | 1.97E−09 |
| Human TGF-β3 | 7.17E+07 | 2.82E−03 | 3.93E−11 |

Note:
NB, no binding, indicates that there was no binding.

### Example 6 In Vivo Anti-Tumor Activity of HB0028

Anti-tumor effect of the antibody in human melanoma A375 combined PBMC subcutaneous xenotransplanted tumor model: Taking 6-8-week-old NCG mice, A375 cells were co-cultured with human PBMCs for 6 days, then the PBMCs and freshly digested A375 cells were collected and mixed in an appropriate ratio, and inoculated subcutaneously into the right sides of the mice at 0.2 ml per mouse. The mice were randomly administered in groups based on their body weight, with the particular administration method, dosage, and administration route shown in Table 2, and the administration started on the day of tumor inoculation which was recorded as day 0. Measurement was performed twice a week using a vernier caliper, and the tumor volume was calculated from a formula of V=0.5 a×b², where a and b represent the long diameter and the short diameter of the tumor, respectively, from which the tumor growth inhibition rate (TGI, %) was calculated.

TABLE 2
Grouping and administration of huPBMC
+ A375 xenotransplanted tumor models

| | | | | | Dosing | Administration |
|---|---|---|---|---|---|---|
| Groups | Dosing group | N | Dosage | | regimen | mode |
| G1 | 900201 (IgG1 | 6 | 25 | mg/kg | BIW × 4 | i.p. |
| | isotype control) | | | | | |
| G2 | M7824 | 6 | 5 | mg/kg | BIW × 4 | i.p. |
| G3 | HB0028 (LD) | 6 | 5 | mg/kg | BIW × 4 | i.p. |
| G4 | HB0028 (HD) | 6 | 25 | mg/kg | BIW × 4 | i.p. |

Note:
N: number of the used animals;
BIW × 4: dosing twice a week, for 4 weeks, in a total of 8 times;
i.p.: intraperitoneal injection

The anti-tumor activity results of the antibodies in the A375 model were shown in FIG. 10. The results showed that, at the same dosage, the inhibitory effect of HB0028 on tumor growth was slightly weaker than that of the control drug M7824 (900544) (P>0.27), while its anti-tumor effect was enhanced at high dosages and comparable to that of the control drug. Compared with the negative control group, various dosing groups can effectively inhibit the tumor growth. At the end of the experiment, the anti-tumor rates of M7824 and HB0028 (high and low dosages) were 78.55%, 76.74% and 58.65%, respectively, with no significant difference among groups (P>0.27). There were no obvious abnormal changes in body weight and preclinical behavior in all groups of mice, indicating that the tumor-bearing mice were well tolerated to each of the tested drugs at the test doses.

Anti-tumor effect of the antibody in the human breast cancer MDA-MB-231 combined PBMC subcutaneous xenotransplanted tumor model:

Taking female NCG mice of 18-22 g, MDA-MB-231 cells were co-cultured with human PBMCs for 6 days, then the PBMCs and freshly digested MDA-MB-231 cells were collected and mixed in an appropriate ratio, and inoculated subcutaneously into the right sides of the mice at 0.2 ml per mouse. After inoculation, when the tumor grew to 70-130 mm³, the mice were randomly divided into 3 groups, 6 in each group, based on the tumor size, with the particular administration method, dosage, and administration route shown in Table 3, and the day of grouping and administration was recorded as day 0.

TABLE 3
Grouping and administration of huPBMC +
MDA-MB-231 xenotransplanted tumor models

| | | | | | Dosing | Administration |
|---|---|---|---|---|---|---|
| Groups | Dosing group | N | Dosage | | regimen | mode |
| G1 | 900201 (IgG1 | 6 | 25 | mg/kg | BIW × 4 | i.p. |
| | isotype control) | | | | | |
| G2 | M7824 | 6 | 5 | mg/kg | BIW × 4 | i.p. |
| G3 | HB0028 | 6 | 5 | mg/kg | BIW × 4 | i.p. |

Note:
N: number of the used animals;
BIW × 4: dosing twice a week, for 4 weeks, in a total of 8 times;
i.p : intraperitoneal injection

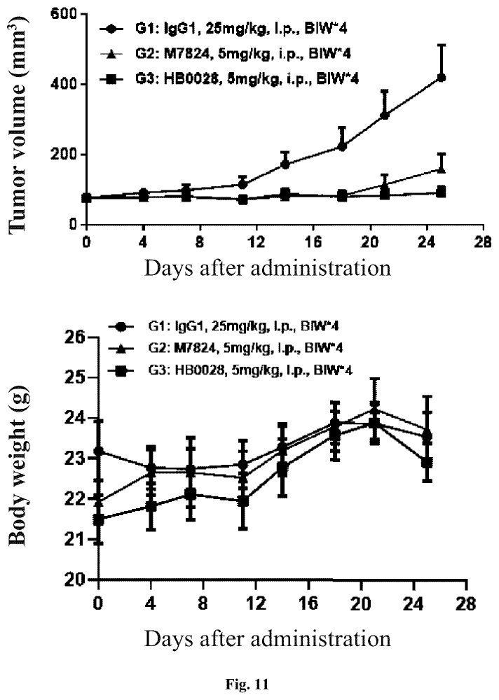

The anti-tumor activity results of the antibodies in the MDA-MB-231 model were shown in FIG. 11. The results showed that, at the same dosage, the inhibitory effect of HB0028 on tumor growth was comparable to that of the control drug M7824, and it even showed a tendency of better tumor suppression than the control drug at the last two doses. At the end of the experiment, compared with the negative control group, the anti-tumor rates of M7824 and HB0028 were 80.16% and 91.52%, respectively. There were no obvious abnormal changes in body weight and preclinical behavior in all groups of mice, indicating that the tumor-bearing mice were well tolerated to each of the tested drugs at the test doses.

### Example 7 Study on the Stability of Fusion Protein

HB0028 and HB0029 samples were exchanged into the same buffer solution and the concentration was adjusted to approximately 1.5 mg/ml. The stability of fusion proteins was evaluated under the above conditions. To compare the thermal stability, a protein stability analyzer (UNcle, UNCHAINED LABS, US) was used to detect the melting temperature (Tm) and the aggregation temperature (Tagg) of two infusion proteins. To compare the protein stability under accelerated and pressurized conditions, the two infusion proteins were placed in a constant temperature incubator at 25° C. at 1 M and 3 M, and in a constant temperature incubator at 40° C. at 1 M. The samples were tested for SEC and CE purity and the changes in purity were compared.

The results shown in Table 4 demonstrated that, the Tm value (68.9° C.) of HB0028 protein was close to the Tm value (69.7° C.) of HB0029 protein, and the Tagg value (69.5° C.) of HB0028 protein was 5° C. higher than the Tagg value (64.2° C.) of HB0029 protein. For acceleration at 25° C. for 3 M, the SEC main peak purity was reduced by 12.3% for HB0028 and 39.5% for HB0029, mainly manifested as the increase of the right shoulder peaks and low molecules (suspected degradation), and no significant difference in non-reduced CE-SDS purity. For being placed at 40° C. for 1 M, the SEC purity was reduced by 13.4% for HB0028 and 24.1% for HB0029, also manifested as the increase of the right shoulder peaks and low molecules. In summary, the thermal aggregation temperature of HB0028 protein was significantly higher than that of HB0029, and the degradation rate under accelerated and high temperature conditions was significantly lower than that of HB0029. Therefore, the molecular structure of HB0028 protein was more stable than that of HB0029.

TABLE 4
Stability results under accelerated and high temperature conditions

| | | UPLC-SEC (%) | | | | | |
|---|---|---|---|---|---|---|---|
| | | High | | | Low | CE-SDS (NR, %) | |
| Sample | Placement | molecular | Main | Shoulder | molecular | Leading | Main |
| Name | conditions | peak | peak | peak | peak | peak | peak |
| HB0028 | T0 | 0.6 | 99.4 | — | — | 4.1 | 95.9 |
| | 25° C., 1 M | 1.3 | 94.4 | 3.2 | 1.1 | 7.9 | 92.1 |
| | 25° C., 3 M | 1.3 | 87.1 | 7.9 | 3.8 | — | — |
| | 40° C., 1 M | 1.6 | 86.0 | 8.8 | 3.8 | 14.1 | 85.9 |
| HB0029 | T0 | 0.7 | 99.3 | — | — | 5.9 | 94.1 |
| | 25° C., 1 M | 1.0 | 82.5 | 14.9 | 1.6 | 8.3 | 91.7 |
| | 25° C., 3 M | 1.7 | 59.8 | 33.1 | 5.4 | — | — |
| | 40° C., 1 M | 2.1 | 75.2 | 20.5 | 2.2 | 13.9 | 86.1 |

All documents mentioned in the present application are incorporated herein by reference as if each document is individually indicated to be incorporated by reference. Additionally, it should be understood that various variations or modifications can be made to the present application by those skilled in the art after reading the above teachings of the present application, and these equivalent forms also fall within the scope defined by the appended claims of the present application. Detection of biological activity of fusion protein in vitro by a reporter gene method.

What is claimed is:

1. A bifunctional antibody, wherein, said bifunctional antibody comprises:
(a) an anti-PD-L1 antibody or element; and
(b) an anti-TGF-β antibody or element connected to said anti-PD-L1 antibody or element.

2. The bifunctional antibody according to claim 1, wherein, said anti-PD-L1 antibody or element is connected to said anti-TGF-β antibody or element through a connecting peptide.

3. The bifunctional antibody according to claim 1, wherein, said anti-TGF-β antibody or element is connected to a region of said anti-PD-L1 antibody selected from the following group: a heavy chain variable region, a heavy chain constant region, a light chain variable region, or a combination thereof.

4. The bifunctional antibody according to claim 1, wherein, said anti-TGF-β antibody or element is connected to an initial terminal of the heavy chain variable region of said anti-PD-L1 antibody.

5. The bifunctional antibody according to claim 1, wherein, said anti-TGF-β antibody or element is connected to a terminal end of the heavy chain constant region of the anti-PD-L1 antibody.

6. (canceled)

7. (canceled)

8. (canceled)

9. The bifunctional antibody according to claim 1, wherein, said element comprises an extracellular region of a ligand, a receptor, or a protein.

10. The bifunctional antibody according to claim 1, wherein, said anti-TGF-β element comprises an extracellular region of a TGF-β receptor; wherein, said TGF-β receptor comprises TGF-βRI, TGF-βRII, and TGF-βRIII; wherein, the number of said anti-TGF-β element is 1 to 4.

11. (canceled)

12. (canceled)

13. (canceled)

14. The bifunctional antibody according to claim 1, wherein, said bifunctional antibody has a structure represented by formula Ia or Ib from N-terminus to C-terminus: 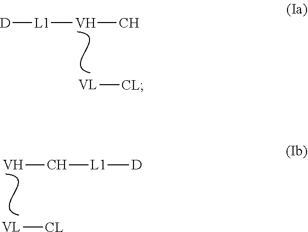
wherein,
“-” represents a peptide bond;
“ [symbol] ” represents a disulfide bond;
D is an anti-TGF-β element;
L1 is none or an adaptor element;
VH represents the heavy chain variable region of the anti-PD-L1 antibody;
CH represents the heavy chain constant region of the anti-PD-L1 antibody;
VL represents the light chain variable region of the anti-PD-L1 antibody;
CL represents the light chain constant region of the anti-PD-L1 antibody;
wherein, said bifunctional antibody has an activity of simultaneously binding to PD-L1 and TGF-β.

15. The bifunctional antibody according to claim 1, wherein, said anti-TGF-β element comprises a TGF-βRII extracellular region; wherein, the amino acid sequence of said TGF-βRII extracellular region is as set forth in SEQ ID NO: 2.

16. (canceled)

17. The bifunctional antibody according to claim 1, wherein, said adaptor element is a GS connecting peptide; wherein, the amino acid sequence of said GS connecting peptide is as set forth in SEQ ID NO: 3.

18. (canceled)

19. (canceled)

20. (canceled)

21. (canceled)

22. (canceled)

23. (canceled)

24. (canceled)

25. The bifunctional antibody according to claim 1, wherein, the heavy chain variable region (VH) of said anti-PD-L1 antibody comprises the following three complementary determining regions (CDRs):
a CDR1 as set forth in SEQ ID NO: 12,
a CDR2 as set forth in SEQ ID NO: 13, and
a CDR3 as set forth in SEQ ID NO: 14.

26. The bifunctional antibody according to claim 1, wherein, the light chain variable region (VL) of said anti-PD-L1 antibody comprises the following three complementary determining regions (CDRs):
a CDR1′ as set forth in SEQ ID NO: 15,
a CDR2′ with an amino acid sequence of GIS, and
a CDR3′ as set forth in SEQ ID NO: 16.

27. The bifunctional antibody according to claim 1, wherein, the amino acid sequence of the heavy chain variable region (VH) of said anti-PD-L1 antibody is as set forth in SEQ ID NO: 4; wherein, the amino acid sequence of the light chain variable region (VL) of said anti-PD-L1 antibody is as set forth in SEQ ID NO: 8.

28. (canceled)

29. (canceled)

30. (canceled)

31. (canceled)

32. (canceled)

33. The bifunctional antibody according to claim 1, wherein, said bifunctional antibody is a homodimer with a structure represented by formula Ia.

34. (canceled)

35. The bifunctional antibody according to claim 1, wherein, said bifunctional antibody has a heavy chain (H chain) and a light chain (L chain); wherein the H chain of said bifunctional antibody has an amino acid sequence as set forth in SEQ ID NO: 1; wherein, the L chain of said bifunctional antibody has an amino acid sequence as set forth in SEQ ID NO: 7.

36. (canceled)

37. (canceled)

38. The bifunctional antibody according to claim 1, wherein, said bifunctional antibody is in a form of a drug conjugate.

39. The bifunctional antibody according to claim 1, wherein, said bifunctional antibody is conjugated with a tumor-targeted marker conjugate, a detectable marker, a targeting label, a drug, a toxin, a cytokine, a radionuclide, and/or an enzyme.

40. (canceled)

41. Isolated polynucleotide, wherein, said polynucleotide encodes said bifunctional antibody of claim 1.

42. (canceled)

43. (canceled)

44. Immunoconjugate, wherein, the immunoconjugate comprises:
(a) the bifunctional antibody of claim 1; and
(b) a conjugating part selected from the following group: a detectable marker, drug, toxin, cytokine, radionuclide, or enzyme, gold nanoparticle/nanorod, nanomagnetic particle, and/or virus coat protein or VLP.

45. A method of preventing and/or treating cancers or tumors comprising administering to a subject in need thereof the bifunctional antibody of claim 1.
